(19) United States
(12) Patent Application Publication
(10) Pub. No.: US 2024/0315559 A1
(43) Pub. Date: Sep. 26, 2024

(54) WEARABLE DEVICE, A SYSTEM AND METHODS FOR FAST ESTABLISHMENT OF A COMMUNICATION CONNECTION FOR A CALL INVOLVING A WEARABLE DEVICE CONNECTED TO A NETWORK

(71) Applicant: Pink Nectarine Health AB, Stockholm (SE)

(72) Inventor: Martin LINREY, Sollentuna (SE)

(73) Assignee: Pink Nectarine Health AB, Stockholm (SE)

(21) Appl. No.: 18/575,398

(22) PCT Filed: Jul. 5, 2022
(86) PCT No.: PCT/SE2022/050683

§ 371 Date: Dec. 29, 2023

(30) Foreign Application Priority Data
Jul. 7, 2021 (SE) 2150905-4

(51) Int. Cl. A61B 5/00; H04W 4/80; H04W 84/18

(52) U.S. Cl. CPC A61B 5/002; A61B 5/6802; A61B 5/7465; H04W 4/80; H04W 84/18

(57) ABSTRACT

Methods, a system, a network of nodes and at least one wearable device. The at least one wearable device is adapted to be worn by individuals and are configured to transmit short-range wireless broadcast signals while a short-range wireless connection between the wearable device and a node of the network of nodes is active. At least one of the network nodes is configured to initiate the establishment of an internal transmission channel or path within the network for routing sound recorded by a microphone of the wearable device as audio data to the network node assigned to receive incoming, to the network, and forward outgoing, from the network, audio data from a conference call or call in which a particular wearable device is to participate.

### TECHNICAL FIELD

The present invention relates to methods, a system, a network of nodes and a wearable personal device. Specifically, the present invention relates to methods for initiating the establishing of an internal transmission channel or path within a network so that a wearable device worn by an individual can participate in a conference call or call using a microphone of the wearable device for recording the speech of the individual.

### BACKGROUND

Systems for monitoring such individuals, e.g. elderly, sick or injured people, and automatically issuing alarms have been used for some time. Monitoring systems for monitoring such individuals often comprise a personal device (e.g. a wristband) comprising sensors, the signals of which are analysed to determine whether help is needed, e.g. due to a fall.

U.S. Pat. No. 6,433,690 discloses a method and system for recording acceleration and body position data from elderly or disabled persons. The fall monitoring system includes signal feature extraction and interpretive methods for characterizing accelerations and body positions during fall events. The system can detect health and life-threatening fall events in elderly persons and can autonomously notify nursing personnel or family members that the person is in need of immediate assistance. The monitoring of a person's fall is performed by using an accelerometer in a monitoring device carried on the person, which monitoring device samples the person's body angle and body acceleration.

U.S. Pat. No. 9,668,297 describes a BLE scatternet system and method that uses a schedule for when advertisements should be transmitted. The advertiser transmits an initial advertisement, receives a connection request, and then goes to sleep to wake up and transmit subsequent advertisements according to the schedule.

U.S. Ser. No. 10/313,866 describes a method for establishing and monitoring a Bluetooth low energy wireless network by using the broadcast state.

US2019/0215244 describes a Self-Organizing Network (SON) for Internet of Things (IoT), where an IoT device may be selected to be an “IoT coordinator”. A coordinator may e.g. perform the roles of managing a neighboring IoT device and forwarding a packet. This SON method includes receiving neighboring device information from at least one neighboring IoT device of an IoT device, updating device information of the IoT device based on the received neighboring device information, and determining whether the IoT device is a coordinator candidate based on at least one of a number of network interfaces, a number of neighbor links connected to the network interfaces, or a ratio of remaining energy, which is included in the device information.

The managing of the IoT devices in US2019/0215244 is performed by neighboring IoT devices and, thus, relies on the presence of other IoT devices within short-range communication range and is also sharing short-range wireless transmission medium and protocol with other IoT devices thus is dependent on the currently available short-range bandwidth of those neighboring IoT devices, e.g. the current number of other links connected to the network interface of those neighboring IoT devices.

There is a need for a more scalable system comprising a network for quickly connecting wearable devices carried by individuals moving between different locations of a facility to a call with a mobile communication device of a caregiver.

More specifically, there is a need for a more scalable system, a network and methods enabling for wearable devices connected to the network and worn by monitored individuals, e.g. patients, to be quickly connected to a call with a mobile communication device of a caregiver and which provides both faster responses to events and obtained data indicating that the wearable device should participate in a call, yet provides for the individuals to be connected to the monitoring network while moving between different locations of a facility covered by the different network nodes of the network

### Problems with the Prior Art

It is difficult to design a system for monitoring wearable devices carried by individuals moving between different locations or rooms of a facility which is highly scalable and provides reliable and fast responses to obtained data which indicates that an individual carrying a wearable device should be connected to a call with its caregiver, yet provides a battery-efficient solution which is robust in that the wearable devices always have a reliable short-range wireless connection to the node network and compete less for bandwidth.

There is thus a need for a scalable network and low-power consuming system for monitoring individuals within a facility that is adapted to provide fast responses to certain events and a change in the behavior or condition of an individual/patient by the establishment of a conference call or call.

### SUMMARY

The technology disclosed provides for a more scalable system comprising a network for quickly and in a robust and battery-efficient connecting wearable devices carried by individuals moving between different locations of a facility to a conference call or call with a mobile communication device of a caregiver.

The technology disclosed relates to methods, a monitoring system, a network of nodes and at least one wearable device. According to the technology disclosed, the at least one wearable device is adapted to be worn by individuals and are configured to transmit short-range wireless broadcast signals while a short-range wireless connection between the wearable device and a node of the network of nodes is active. At least one of the network nodes is configured to initiate the establishment of an internal transmission channel or path within the network for routing sound recorded by a microphone of the wearable device as audio data to the network node assigned to receive incoming, to the network, and forward outgoing, from the network, audio data from a conference call or call in which a particular wearable device is to participate.

In aspects, the technology disclosed provides for a system and self-organizing network adapted for monitoring wearable devices carried by individuals moving between different locations or rooms of a facility which is highly scalable and provides reliable and fast responses to obtained data which indicates that an individual carrying a wearable device should be connected to a call with its caregiver yet provides a battery-efficient solution which is robust in that the wearable devices always have a reliable short-range wireless connection to the node network and in that the wearable devices compete less for bandwidth.

The technology disclosed relates to a scalable network and low-power consuming system for monitoring individuals within a facility that is adapted to provide fast responses to certain events and a change in the behaviour or condition of an individual/patient by the establishment of a conference call or call in which the individual/patient carrying a wearable device connected to the network may participate.

More specifically, the technology disclosed provides for a more scalable system, a network and methods enabling for wearable devices connected to the network and worn by monitored individuals, e.g. patients, to be quickly connected to a call with a mobile communication device of a caregiver and which provides both faster responses to events and obtained data indicating to the network nodes that the wearable device should participate in a call, yet provides for the individuals to be connected to the monitoring network while moving between different locations of a facility covered by the different network nodes of the network.

In aspects and embodiments, the technology disclosed relates to a system for monitoring individuals including a backend system and a self-organizing network (SON) comprising:
at least three network nodes including at least two network nodes comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means, wherein each of the at least one wearable device is configured to establish a short-range wireless communication link with one of the network nodes and is assigned a network node configured to, in response to receiving downlink data from the backend system indicating that a conference call or call should be established with the wearable device, initiate the establishment of an internal transmission channel or path within the SON for routing sound recorded by the microphone of the wearable device as audio data to the network node and for routing to the gatherer node incoming audio data of a conference call or call from the network node to another network node so that the audio data can be played out by the speaker of the other network node.

In aspects and embodiments, the technology disclosed relates to a system for monitoring individuals including a backend system and a self-organizing network (SON) comprising:
at least three network nodes including at least two network nodes comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means, wherein a wearable device is configured to establish a short-range wireless communication link with one of the network nodes and is assigned a network node configured to, in response to receiving downlink data from the backend system indicating that a conference call or call should be established with the wearable device, initiate the establishment of an internal transmission channel or path within the SON for routing sound recorded by the microphone of the wearable device as audio data to the network node and for routing to the gatherer node incoming audio data of a conference call or call from the network node to another network node so that the audio data can be played out by the speaker of the other network node.

In embodiments, the backend system is configured to transmit the downlink data to the network node assigned to the wearable device in response to receiving uplink data from the SON indicating to the backend system at least one of an alert state, a changed physical condition, a changed health state, a changed physical orientation of the person wearing the wearable device and that a call should be established with the wearable device.

In embodiments, the downlink data received by the network node assigned to the wearable device is included in at least one of a configuration file which is continuously updated by the backend system and transmitted to the SON, a call initiation request including data for identifying a wearable device which is to participate in a conference call or call, and control data indicating to the network node assigned to the wearable device that the wearable device is to participate in a conference call or call.

In embodiments, the backend system is configured to continuously update data associated with the wearable device based on input data and/or sensor data received by the backend system from the SON, and wherein the downlink data which may trigger the initiation of an internal transmission channel or path within the SON is included in a configuration file continuously updated and transmitted by the backend system to the SON.

In embodiments, the network node assigned to the wearable device is configured to initiate the establishment of an internal transmission channel or path within the SON for the purpose of establishing a conference call or call in which the wearable device is to participate in response to the updated and transmitted configuration file is indicating to the network node at least one of an alert state, a changed physical condition, a changed health state, a changed physical orientation of the person wearing the wearable device and that a call should be established with the wearable device.

In embodiments, the network node assigned to the wearable device is configured to initiate the establishment of an internal transmission channel or path to a network node having an established short-range wireless communication link with the wearable device where the internal transmission channel or path includes at least one communication link between the network node and another network node. In certain embodiments, the network node assigned to the wearable device is configured to use the same internal routing table for initiating the establishment of the internal transmission channel or path for routing audio data obtained by the microphone of the identified wearable device from the other network node to the network node as for the routing of incoming audio data of a conference call or call to the SON from the network node to the other network node for play out by the speaker of the other network node.

In aspects, the technology disclosed relates to a method for monitoring individuals in a system including a backend system, a self-organizing network (SON) comprising at least three network nodes including at least two network nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. assigning one of the network nodes to a wearable device;
b. transmitting, by the backend system, downlink data to the SON, and;
c. receiving, by the network node assigned to the wearable device, said downlink data from the backend system; and
d. initiating, by the network node assigned to the wearable device and in response to the downlink data from the backend system is indicating to the network node assigned to the wearable device that the wearable device is to participate in a conference call or call, the establishment of an internal transmission channel or path within the SON for routing sound recorded by the microphone of the wearable device as audio data to the network node assigned to the wearable device and for routing to the network node incoming audio data of a conference call or call from the network node assigned to the wearable device to one of the other network nodes so that the audio data can be played out by the speaker of the other network node.

In embodiments, the method is further comprising:
forwarding, by the network node assigned to the wearable device, audio data recorded by the microphone of the wearable device and transmitted over the internal transmission channel or path within the SON to at least one of an external system for managing the conference call or call in which the wearable device and a mobile communication device associated with the wearable device is participating and a mobile communication device associated with the wearable device.

In embodiments, the method is further comprising:
transmitting, by the network node assigned to the wearable device and in response to the downlink data is indicating that a call should be established with the wearable device, control data for activating the microphone of the wearable device.

In embodiments, the method is further comprising:
determining, by the network node assigned to the wearable device and based on or in response to the downlink data is indicating that a call should be established with the wearable device, which of the at least three nodes is to activate its speaker to play out audio data received from the conference call or call routed over the internal transmission channel or path.

In embodiments, the determining by the gatherer node which of the network nodes is to activate its speaker to play out audio data received from the conference call or call is at least partly based on at least one of signal strength values of short-range wireless signals transmitted by the wearable device and which are measured by at least one of the network nodes, and data the wearable device which is collected by at least one other network node and then shared with the other hub nodes by distribution within the SON.

In embodiments, the method is further comprising:
receiving, by the backend system and from the SON, at least one of input data obtained by the user input means of the wearable device and sensor data obtained by at least one sensor of the wearable device; and
transmitting, by the backend system and at least partly based on the received input data and/or sensor data obtained by the user input means and/or at least one sensor of the wearable device, the downlink data to the SON.

In embodiments, the at least one of input data and sensor data obtained by the wearable device is transmitted to the backend system by the network node assigned to have the controller node role for the wearable device, wherein said controller node is responsible for determining and dynamically changing which of the nodes of the SON is currently having an established short-range wireless connection with the wearable device.

In embodiments, the method is further comprising:
initiating, by the backend system and in response to uplink data received from the SON is indicating to the backend system that a conference call or call should be established with the wearable device, a conference call or call in which the wearable device and a mobile communication device linked to the wearable device in the backend system are to participate.

In embodiments, the method is further comprising initiating, by the backend system, the establishment of a conference call or call by transmitting a request to a third-party call facilitator requesting the third-party call facilitator to start a conference call or call in which the wearable device and a mobile communication device linked to the wearable device in the backend system can call into.

In embodiments, the method is further comprising initiating, by the backend system, the establishment of a conference call or call by transmitting a request to the network node assigned to the wearable device requesting the network node to activate its two-way audio client to call into the conference call or call.

In aspects and embodiments, the technology disclosed relates to a system for monitoring individuals including a backend system, a self-organizing network (SON) comprising:
at least three network nodes including at least one hub node,
at least two satellite nodes each comprising a speaker and configured to transmit short-range wireless broadcasts and to establish a short-range wireless communication link with a wearable device, and at least one wearable device having short-range wireless communication capabilities and comprising a microphone and at least one of at least one sensor and a user input means, and
wherein each of the at least one wearable device is having an established short-range wireless communication link with one of the network nodes and is assigned a hub node to have the role of a gatherer node for the wearable device, and wherein the hub node having the gatherer node role for a wearable device is configured to, in response to receiving downlink data from the backend system indicating that a conference call or call should be established with a wearable device the network node is the gatherer node for, initiate the establishment of an internal transmission channel or path within the SON for routing sound recorded by the microphone of the wearable device as audio data to the gatherer node and for routing to the gatherer node incoming audio data of a conference call or call from the gatherer node to another network node so that the audio data can be played out by the speaker of the other network node.

In embodiments of the technology disclosed, the above-identified problems are addressed by the claimed system for monitoring individuals within predetermined facilities by providing a network of nodes, e.g. a self-organizing network (SON), including at least one network node assigned to a particular personal device and configured for initiating a transmission path or channel to be used for a conference call or call involving the particular personal device, e.g. a wearable device, and a mobile communication device associated with the personal device.

The technology disclosed relates to a wearable device, a system and methods for fast establishment of a communication connection for an externally established call or conference call involving a wearable device connected to a network node of a self-organizing network via a short-range wireless connection and which is connected to different network nodes while moving between different locations of a facility in which the different network nodes have network coverage in different locations of the facility.

In aspects, the at least one network node of the network, e.g. SON, is configured to initiate the establishment of an internal transmission channel or path within the SON for routing sound recorded by a microphone of a wearable device.

In aspects, the internal transmission channel or path initiated by the network node may also be used for routing to the network, e.g. SON, incoming audio data of a conference call or call from the network node to another network node of the network so that the audio data of the conference call or call can be played out by the speaker of the other network node.

The technology disclosed relates to methods, a system comprising a network of nodes, e.g. a self-organizing network (SON), and at least one wearable device each configured to transmit data associated with the respective wearable device both via an established short-range wireless connection links with one of the network nodes of the node network and via short-range wireless broadcasts which may be received by any of the network nodes that are within short-range wireless communication range with the wearable device.

The technology disclosed also relates to a system and methods for transmitting data associated with a personal device worn by an individual both via an established short-range wireless connection link between the personal device and a network node and via short-range wireless broadcasts from the personal device.

In aspects, the personal device is adapted to be worn by an individual and is configured to transmit short-range wireless broadcast signals while a short-range wireless connection between the wearable device and a node of the network of nodes is active, thereby being configured for providing its obtained input data and/or sensor data to other nodes of the network that are within short-range wireless communication range with the personal device.

In aspects, one of the hub nodes is assigned to have the controller node role for a wearable device and be responsible for collecting input data and/or sensor data obtained by the wearable device. The input data and/or sensor data obtained by the wearable device may be received in broadcast messages transmitted by the wearable device if the controller node is within short-range wireless communication range with the wearable device or may be received from other network nodes within the SON, including satellite nodes which are configured to both transmit short-range wireless broadcast signals and listen for short-range wireless broadcast signals transmitted by other devices and nodes.

In embodiments, each of the at least one wearable device is assigned one of the at least one hub node to have the controller node role for the wearable device and be configured for dynamically determining and changing which node is currently having a short-range wireless communication link established with the wearable device.

In embodiments, the hub nodes of the SON are communicatively connected to each other via an IP-based network such as a Wi-Fi and each of the hub nodes are configured to share data associated with any of the wearable devices connected to a node of the SON via continuous distribution of the data within the IP-based network.

In embodiments, the hub nodes of the SON are communicatively connected to each other via a network of established short-range wireless communication links established between the hub nodes, e.g. established Bluetooth/BLE connections, and each of the hub nodes are configured to share data associated with any of the wearable devices connected to a node of the SON via continuous distribution of the data within the network of established short-range wireless communication links, e.g. BLE links.

In aspects, each of the satellite nodes may have an established short-range wireless communication link, e.g. BLE link, with one of the wearable devices, where the establishment of the short-range wireless communication link is initiated by the hub node of the SON which is the controller node for the wearable device.

In aspects, the hub node having the controller node role for a wearable device is also responsible for assigning one of the hub nodes, including itself, to have the gatherer node role for the wearable device. The hub node having the gatherer node role for a certain wearable device is configured to be managing audio data in externally established calls and/or conference call and be responsible for incoming and outgoing audio data of conference calls and/or calls in which the wearable device is participating.

The technology disclosed relates to a method and a system including a backend system, a network comprising at least three network nodes where at least two of the network nodes comprise a speaker, and at least one wearable device having short-range wireless communication capabilities and comprising a microphone and at least one of at least one sensor and user input means,
wherein each of the at least one wearable device is having an established short-range wireless communication link with one of the network nodes and is assigned one of the network nodes to receive incoming and forward outgoing audio data from a conference call or call in which a particular wearable device is to participate, and wherein the network node assigned to receive incoming and forward outgoing audio data from a conference call or call in which the particular wearable device is to participate, is configured to, in response to receiving downlink data from the backend system indicating that a conference call or call should be established with the wearable device, initiate the establishment of an internal transmission channel or path within the network for routing sound recorded by the microphone of the wearable device as audio data to the network node assigned to receive incoming and forward outgoing audio data from a conference call or call in which the particular wearable device is to participate.

In embodiments, the technology disclosed relates to a method and a system including a backend system, a network comprising at least three network nodes where at least two of the network nodes comprise a speaker, and at least one wearable device having short-range wireless communication capabilities and comprising a microphone and at least one of at least one sensor and user input means,
wherein each of the at least one wearable device is having an established short-range wireless communication link with one of the network nodes and is assigned one of the network nodes to receive incoming and forward outgoing audio data from a conference call or call in which a particular wearable device is to participate, and wherein the network node assigned to receive incoming and forward outgoing audio data from a conference call or call in which the particular wearable device is to participate, is configured to, in response to receiving downlink data from the backend system indicating that a conference call or call should be established with the wearable device, initiate the establishment of an internal transmission channel or path within the network for routing sound recorded by the microphone of the wearable device as audio data to the network node assigned to receive incoming and forward outgoing audio data from a conference call or call in which the particular wearable device is to participate, wherein the internal transmission channel or path is established via at least one already established short-range wireless communication link.

In embodiments, the transmission of audio data recorded by the microphone over the internal transmission channel or path includes the transmission of audio data over at least one established Bluetooth link, e.g. BLE links. In certain embodiments, the transmission of audio data over the internal transmission channel or path includes the transmission of audio data between at least one established Bluetooth communication links between network nodes and/or the established Bluetooth communication link between the connection node for the wearable device and the wearable device.

In embodiments, the network node assigned to be receiving incoming, to the network, and forward outgoing, from the network, audio data from a conference call or call in which the particular wearable device is to participate, is configured to, in response to receiving downlink data from the backend system indicating that a conference call or call should be established with the wearable device, initiate the establishment of an internal transmission channel or path within the network for routing sound recorded by the microphone of the wearable device as audio data to the gatherer node and for routing to the gatherer node incoming audio data of a conference call or call from the gatherer node to another network node so that the audio data can be played out by the speaker of the other network node. In embodiments, the transmission of audio data over the internal transmission channel or path includes the transmission of audio data over established Bluetooth link, e.g. BLE links. In certain embodiments, the transmission of audio data over the internal transmission channel or path includes the transmission of audio data between established Bluetooth communication links between the network nodes and/or the established Bluetooth communication link between the connection node for the wearable device and the wearable device.

In embodiments, the downlink data received by the network from the backend system is based on uplink data in the form of at least one of input data and sensor data received from the network, e.g. received from certain network nodes responsible for transmitting input data and/or sensor data obtained by individual wearable devices they are responsible for.

In embodiments, the hub node having the gatherer node role for a certain wearable device is further configured to determine which of the network nodes is to activate its speaker to play audio data received from the gatherer node over the internal transmission channel or path within the network.

In embodiments, the hub node having the gatherer node for a wearable device is further configured to determine which of the network nodes is to activate its speaker to play out audio data received from the conference call or call routed over the internal transmission channel or path at least partly based on at least one of signal strength values of short-range wireless signals transmitted by the wearable device and which are measured by at least one of the at least two satellite nodes and then received by the gatherer node via short-range wireless transmissions from the at least one of the at least two satellite nodes, and signals strength values and/or data related to the wearable device which is collected by at least one other hub node and then shared with the gatherer node by distribution within the SON.

In embodiments, the backend system is configured to determine that a conference call or call in which the wearable device is to participate is to be established based on the input data and/or sensor data received from the network, e.g. a network node assigned to be responsible for the wearable device.

In the embodiments, the decision to establish a call and the decision which of the network nodes is to play out audio data from the call through its speaker may be based at least partly on received sensor data, e.g. obtained by a temperature sensor, pressure sensor or motion sensor of the wearable device. In various embodiments, the actual decision to establish a conference call or call may then be taken by a backend system or the network node having the controller node role for the wearable device. The determining which of the network nodes is to play out incoming audio data from the conference call or call transmitted over the internal channel or path through its speaker may be taken by the network node having a gatherer node role for the wearable device or the controller node for the wearable device which informs the gatherer node about its decision by transmitting certain data to the gatherer node.

In embodiments, the downlink data received by the network from the backend system is based on uplink data from the network indicating to the backend system at least one of an alert state, a changed physical condition, a changed health state, a changed physical orientation of the person wearing the wearable device and that a call should be established with the wearable device.

In embodiments, the backend system is configured to transmit downlink data in response to receiving certain uplink data from the SON in that the backend system is configured to transmit downlink data in response to receiving certain uplink data including uplink data indicating at least one of an alert state, a changed physical condition, a changed health state, a changed physical orientation of the person wearing the wearable device and that a call should be established with the wearable device. Hence, for some embodiments of the technology disclosed, the backend system is configured to transmit the downlink data only in response to receiving certain uplink data indicating at least one of an alert state and that a call should be established with the wearable device but is not configured to transmit the downlink data in response receiving uplink data indicating a changed health state or a changed physical orientation of the person wearing the wearable device. For other embodiments of the technology, the backend system is configured to transmit the downlink data in response to certain uplink data is indicating that a call should be established with the wearable device but is not configured to transmit the downlink data in response receiving uplink data indicating an alert state, a changed health state and a changed physical orientation of the person wearing the wearable device. According to embodiments and aspects of the technology disclosed, the hub node having the gatherer node role for the wearable device to which the certain uplink data received by the backend system concerns may then be configured to, in response to the downlink data from the backend system is indicating to the gatherer node that the wearable device should participate in a conference call or call, initiate the establishment of an internal transmission channel or path within the SON for routing sound recorded by the microphone of the wearable device as audio data to the node having the gatherer node role for the wearable device and for routing to the gatherer node incoming audio data of a conference call or call from the gatherer node to one of the other network nodes so that the audio data can be played out by the speaker of the other network node.

In embodiments, the downlink data received by the network, e.g. a network node assigned to manage conference calls or calls for a particular wearable device, is included in at least one of a configuration file which is continuously updated by the backend system and transmitted to the network, a call initiation request including data for identifying a wearable device which is to participate in a conference call or call, and control data indicating to the network that the wearable device is to participate in a conference call or call.

In embodiments, the backend system is configured to continuously update data associated with the at least one wearable device based on input data and/or sensor data received by the backend system from the network, wherein the downlink data triggering the initiation of an internal transmission channel or path within the network is included in a configuration file continuously updated by the backend system and then transmitted by the backend system to the network.

In embodiments, one of the network nodes assigned to manage conference calls and calls for a wearable device is configured to initiate the establishment of an internal transmission channel or path within the network for the purpose of establishing a conference call or call in which the wearable device is to participate in response to the updated and transmitted configuration file is indicating to the network node at least one of an alert state, a changed physical condition, a changed health state, a changed physical orientation of the person wearing the wearable device and that a call should be established with the wearable device.

In embodiments, the downlink data transmitted by the backend system to the network is based on input data and/or sensor data obtained by the wearable device and received from the network, and wherein the backend system is configured to determine that a conference call or call in which the wearable device is to participate is to be established based on the input data and/or sensor data received from the network, e.g. a network node assigned to be responsible for the wearable device. The downlink data transmitted by the backend system to the network node may then be adapted to indicate to the network nodes receiving the downlink data, or data based on the downlink data, that a conference call or call should be established with the wearable device. A network node responsible for managing conference call or calls for the wearable device may then be configured to initiate an internal transmission channel or path based on or in response to the downlink data received from the backend system, or in response to receiving further downlink data from the backend system, e.g. second downlink data in the form of a call initiation request which follows first downlink data in the form of a configuration file which data is indicating to the network node that a conference call or call should be established with the wearable device.

In embodiments, the network node managing conference calls and calls for a particular wearable device is configured to use the same internal routing table for initiating the establishment of the internal transmission channel or path for routing audio data obtained by the microphone of the identified wearable device from the another network node to the network node and for routing incoming audio data of a conference call or call to the network from the network node to the other network node for play out by the speaker of the other network node.

In embodiments, the network node managing conference calls and calls for a particular wearable device is configured to determine which one of the network nodes is to receive the audio data incoming to the network through its speaker based on at least one of signal strength values of wireless short-range signals transmitted by the wearable device which and which are measured by other network nodes within short-range communication range with the wearable device.

In embodiments, the network node managing conference calls and calls for a particular wearable device is configured to determine that the one network node currently having an established wireless short-range communication link with the wearable device is the network node for receiving incoming audio data over the internal transmission channel or path and be playing out audio data through its speaker. In certain embodiments, data indicating to network node which of the network nodes is currently having an established wireless short-range communication link with the wearable device is received by the network node managing conference calls and calls for the wearable device from another network node assigned to be responsible for the wearable device.

In embodiments, the network node currently having an established wireless short-range communication link with a wearable device is configured to at least one of transmit and forward a request received from the network node managing conference calls and calls for the wearable device to the wearable device requesting the wearable device to activate its microphone and then transmit sound data recorded by the microphone as audio data to the network node currently having an established wireless short-range communication link with the wearable device. The audio data is then forwarded, by the network node currently having an established wireless short-range communication link with the wearable device, to the network node managing conference calls and calls for the wearable device which, in turn, forwards the audio data as outgoing audio data from the network, e.g. to an external system managing the conference call or call.

In embodiments, the backend system is configured to initiate the establishment of a conference call or call in which the wearable device is participating and transmit downlink data, e.g. a call initiation requesting, in response to receiving uplink input data, alert/alarm data and/or sensor data from the network. In embodiments, the backend system may be configured to determine that a conference call or call should be established with the wearable device and a mobile communication device associated with the wearable device at least partly based on input data, alert/alarm data and/or sensor data received from the network. In embodiments, the uplink data may indicate to the backend system at least one of an alert state, a changed physical condition, a changed health state, a changed physical orientation of the person wearing the wearable device and that a call should be established with the wearable device.

In embodiments, the backend system is configured to transmit a call initiation request to the network/gatherer node which comprises at least one of a calling number and SIP credentials (e.g. SIP address and/or SIP username), wherein the network node having the role of a gatherer node for a wearable device is configured to, in response to receiving the call initiation request from the backend system, activate its audio client to call into a conference and/or call using the at least one of a calling number and SIP credentials.

In embodiments, the downlink data from the backend system is comprised in a call initiation request which comprises at least one of a calling number and SIP credentials (e.g. SIP address and/or SIP username), and wherein the network node managing conference calls or calls for a wearable device is configured to, in response to receiving the call initiation request from the backend system, activate its audio client to call into the conference and/or call using the at least one of a calling number and SIP credentials.

In embodiments, each of the wearable devices is configured to receiving a request from the network node currently having an established wireless short-range communication link with the wearable device and, in response to receiving the request, activate its microphone to start recording sound data and transmit recorded sound data as audio data to the network node so that the audio data is transmitted over the internal transmission channel or path within the network to the network node managing the conference call or call for the wearable device.

In embodiments, the technology disclosed relates to a method and a system including a backend system, a network comprising at least three network nodes where at least two of the network nodes comprise a speaker, and at least one wearable device having short-range wireless communication capabilities and comprising a microphone and at least one of at least one sensor and user input means,
wherein each of the at least one wearable device is having an established short-range wireless communication link with one of the network nodes and is assigned one of the network nodes to receive incoming and forward outgoing audio data from a conference call or call in which a particular wearable device is to participate, and wherein the network node assigned to receive incoming and forward outgoing audio data from a conference call or call in which the particular wearable device is to participate, is configured to, in response to receiving downlink data from the backend system indicating that a conference call or call should be established with the wearable device, initiate the establishment of an internal transmission channel or path within the network for routing sound recorded by the microphone of the wearable device as audio data to the network node assigned to receive incoming and forward outgoing audio data from a conference call or call in which the particular wearable device is to participate, wherein the internal transmission channel or path is established via at least one already established Bluetooth link, e.g. BLE link.

In embodiments, the technology disclosed relates to a system including a backend system, a self-organizing network (SON) comprising:
at least three network nodes including at least one hub node and at least two satellite nodes each comprising a speaker and configured to transmit short-range wireless broadcasts and to establish a short-range wireless communication link with a wearable device, and
at least one wearable device having short-range wireless communication capabilities and comprising a microphone and at least one of at least one sensor and user input means,

wherein each of the at least one wearable device is having an established short-range wireless communication link with one of the network nodes and is assigned a hub node to have the role of a gatherer node for the wearable device, and wherein the hub node having the gatherer node role for a wearable device is configured to, in response to receiving downlink data from the backend system indicating that a conference call or call should be established with a wearable device the network node is the gatherer node for, initiate the establishment of an internal transmission channel or path within the SON for routing sound recorded by the microphone of the wearable device as audio data to the gatherer node.

In embodiments, the technology disclosed relates to a system including a backend system, a self-organizing network (SON) comprising:
at least three network nodes including at least one hub node and at least two satellite nodes each comprising a speaker and configured to transmit short-range wireless broadcasts and to establish a short-range wireless communication link with a wearable device, and
at least one wearable device having short-range wireless communication capabilities and comprising a microphone and at least one of at least one sensor and user input means,

wherein each of the at least one wearable device is having an established short-range wireless communication link with one of the network nodes and is assigned a hub node to have the role of a gatherer node for the wearable device, and wherein the hub node having the gatherer node role for a wearable device is configured to, in response to receiving downlink data from the backend system indicating that a conference call or call should be established with a wearable device the network node is the gatherer node for, initiate the establishment of an internal transmission channel or path within the SON for routing both sound recorded by the microphone of the wearable device as audio data to the gatherer node and for routing to the gatherer node incoming audio data of a conference call or call from the gatherer node to another network node so that the audio data can be played out by the speaker of the other network node.

In embodiments, the backend system is configured to transmit the downlink data in response to receiving uplink data from the SON indicating to the backend system at least one of an alert state, a changed physical condition, a changed health state, a changed physical orientation of the person wearing the wearable device and that a call should be established with the wearable device.

In embodiments, the downlink data received by the SON and the gatherer node is included in at least one of a configuration file which is continuously updated by the backend system and transmitted to the SON, a call initiation request including data for identifying a wearable device which is to participate in a conference call or call, and control data indicating to the hub node having the gatherer node role for a wearable device that the wearable device is to participate in a conference call or call.

In embodiments, the backend system is configured to continuously update data associated with the at least one wearable device based on input data and/or sensor data received by the backend system from the SON, and wherein the downlink data which may trigger the initiation of an internal transmission channel or path within the SON is included in a configuration file continuously updated and transmitted by the backend system to the SON.

In embodiments, the hub node assigned to have the gatherer node role for a wearable device is configured to initiate the establishment of an internal transmission channel or path within the SON for the purpose of enabling a conference call or call in which the wearable device is to participate in response to the updated and transmitted configuration file is indicating to the gatherer node at least one of an alert state, a changed physical condition, a changed health state, a changed physical orientation of the person wearing the wearable device and that a call should be established with the wearable device. The hub node having the gatherer node role for the wearable device may be configured to determine which of the other network nodes and itself is to receive audio data incoming to the gatherer node from the conference call or call over the internal transmission channel or path within the SON and emit the externally-generated audio data through its speaker. The other network node emitting the audio data from the conference call or call may then be a satellite node and the hub node having the role of a gatherer node for a wearable device may be configured to initiate the establishment of an internal transmission channel or path to the satellite node which includes at least one short-range wireless communication link between the satellite node and another network node.

In embodiments, the network node having the gatherer node role for a wearable device is configured to use the same internal routing table for initiating the establishment of the internal transmission channel or path for routing audio data obtained by the microphone of the identified wearable device from the other network node to the gatherer node and for routing incoming audio data of a conference call or call to the SON from the gatherer node to the other network node for play out by the speaker of the other network node.

In embodiments, the network node having the gatherer node role for a wearable device may be configured to determine which one of the network nodes is to receive the audio data incoming to the SON through its speaker based on at least one of signal strength values of wireless short-range signals received from the wearable device which are measured by other network nodes and data shared with the gatherer node role by the hub node having the controller node role for the wearable device.

In embodiments, the network node having the gatherer node role for a wearable device may be configured to determine that the one network node currently having an established wireless short-range communication link with the wearable device is also the network node for receiving incoming audio data over the internal transmission channel or path and be playing out the audio data from the conference call or call through its speaker.

In embodiments, the network node currently having an established wireless short-range communication link with the wearable device may be configured to at least one of transmit and forward a request to the wearable device requesting the wearable device to activate its microphone and then transmit sound recorded by the microphone as audio data to the network node over the already established wireless short-range communication link with the wearable device.

In embodiments, the backend system is configured to initiate the establishment of a conference call or call in which the wearable device is to be participating by transmitting downlink data, e.g. a call initiation request or an updated configuration file, in response to receiving uplink data from the SON indicating to the backend system at least one of an alert state, a changed physical condition, a changed health state, a changed physical orientation of the person wearing the wearable device and that a call should be established with the wearable device. The uplink data may then be transmitted to the backend system from the one network node having the controller node role for the wearable device.

In embodiments, the downlink data transmitted by the backend system may be included e.g. in a call initiation request which comprises at least one of a calling number and SIP credentials (e.g. SIP address and/or SIP username), and wherein the network node having the role of a gatherer node for a wearable device may be configured to, in response to receiving the call initiation request from the backend system, activate its audio client to call into a conference and/or call using the at least one of a received calling number and received SIP credentials.

In embodiments, each of the wearable devices may be configured to receiving a request from the network node currently having an established wireless short-range communication link with the wearable device and, in response to receiving the request, activate its microphone to start recording sound data and transmit recorded sound data as audio data to the network node so that the audio data is transmitted over the internal transmission channel or path within the SON to the network node having the gatherer node role for the wearable device. The hub node having the gatherer node role for the wearable device may then forward the audio data received from the wearable device over the internal transmission channel or path to an external third-party system/facilitator managing the conference call or call.

In embodiments, the network node currently having an established wireless short-range communication link with the wearable device is configured to, in response to receiving a request from the gatherer node, activate its speaker so that incoming audio data from the conference call or call which is transmitted over the internal transmission channel or path is emitted or played out by the speaker of the network node currently having an established wireless short-range communication link with the wearable device.

In embodiments, the hub node having the gatherer node role for a wearable device is further configured to forward the audio data recorded by the microphone and received over the internal transmission channel or path within the SON to at least one of an external system managing the conference call or call in which an external mobile communication device associated with the wearable device is participating and a mobile communication device associated with the wearable device which is participating in a call with the wearable device.

In embodiments, the hub node having the gatherer node role for a wearable device is further configured to, in response to receiving downlink data from the backend system which indicates at least one of an alert state, a changed physical condition, a changed health state, a changed physical orientation of the person wearing the wearable device and that a call should be established with the wearable device, transmit control data for instructing the activation of the microphone of the wearable device and/or control data for instructing the activation of the speaker of the network node determined to play out the audio data incoming to the gatherer node. The network node determined to be receiving and playing out the incoming audio data may then be a satellite node the wearable device is currently having an established wireless short-range communication link with. The satellite node may then be further configured to transmit a request, or forward a request received from the gatherer node, to the wearable device instructing the wearable device to activate its microphone and then start transmitting audio data obtained by the microphone over the wireless short-range communication link.

In embodiments, the backend system is configured to transmit the downlink data to the SON and the gatherer node at least partly based on at least one of input data obtained by the user input means of the wearable device and sensor data obtained by at least one sensor of the wearable device and which is received by the backend system from the SON. In embodiments, the at least one of input data and sensor data obtained by the wearable device is transmitted to the backend system by the one hub node having the controller node role for the wearable device. The controller node is responsible for determining and dynamically changing which of the nodes of the SON is currently having the only established short-range wireless connection between the wearable device and the SON.

In embodiments, the downlink data received from the backend system is included in at least one of a configuration file which is continuously updated, by the backend system based on sensor data and/or sensor data received by the backend system from the SON/the controller nodes, and then transmitted from the backend system to the SON. The configuration file may then be transmitted as control data transmitted downlink from the backend system to the SON. In various embodiments of the technology disclosed, the control data received from the backend system may in itself be adapted for indicating to a controller node for a wearable device that the wearable device should connect to a conference call or call or the configuration file may only provide the controller nodes with continuously updated sensor data and/or input data and the network node having the controller node role for a wearable device may be configured to determine, based on the updated sensor data and/or input data in the configuration file, that the wearable device should connect to a conference call or call. A network node having the controller node role for a wearable device may then be further configured to transmit control data to the wearable device instructing the wearable device to activate its microphone and start recording sound data that is transmitted uplink to the gatherer node for the wearable device as audio data which is forwarded externally by the gatherer node to the conference call or call.

In embodiments, the downlink data is received by the one hub node having the gatherer node role for the wearable device. The hub node having the gatherer node for a wearable device may be further configured to determine which of the network nodes is to activate its speaker to play out audio data received from the conference call or call routed over the internal transmission channel or path at least partly based on at least one of signal strength values of short-range wireless signals transmitted by the wearable device, including short-range wireless broadcast signals, and which are measured by at least one of the at least two satellite nodes and then received by the gatherer node via short-range wireless transmissions from the at least one of the at least two satellite nodes, and signals strength values and/or data related to the wearable device which is collected by at least one other hub node and then shared with the gatherer node by distribution within the SON.

In embodiments, the hub node having the gatherer node for a wearable device may be further configured to determine which of the nodes is to activate its speaker to play out the incoming audio data from the conference call or call and which is routed over the internal transmission channel or path at least partly based on obtained or received data indicating which of the nodes is the only node in the SON currently having an established short-range wireless connection with the wearable device.

In embodiments, the downlink data is received by the one hub node assigned by the SON to have the controller node role for the wearable device and responsible for determining and dynamically changing which only one of the nodes of the SON is currently having an established short-range wireless connection with the wearable device, and the downlink data is then shared with the hub node having the gatherer node role for the wearable device.

In embodiments, each of the at least one wearable device is assigned one of the at least one hub node to have the controller node role for the wearable device and be configured for dynamically determining and changing which network node is currently having a short-range wireless communication link established with the wearable device.

In embodiments, the at least one hub node having the gatherer node role for a certain wearable device is further configured to determine which of the network nodes in the SON is to activate its speaker to play audio data received from the gatherer node over the internal transmission channel or path within the SON.

In embodiments, each of the at least one hub node is configured to continuously receive an updated configuration file from the backend system including status data for each of the at least one wearable device, and wherein the hub node having the gatherer node role for a wearable device is configured to initiate the establishment of an internal transmission channel or path within the SON in response to the updated status data for the wearable device is indicating that the wearable device is to participate in a conference call or call In embodiments, the backend system is configured to, in response to uplink data received from the SON is indicating to the backend system that a conference call or call should be established with one of the at least one wearable device, initiate the establishment of a conference call or call in which the wearable device and a mobile communication device linked to the wearable device in the backend system are to participate.

In embodiments, the backend system is configured to initiate the establishment of a conference call or call by transmitting a request to a third-party call facilitator requesting the third-party call facilitator to start a conference call or call in which the wearable device and a mobile communication device linked to the wearable device in the backend system can call into.

In embodiments, the backend system is configured to initiate the establishment of a conference call or call by transmitting a request to the hub node having the gatherer node role for a wearable device requesting the gatherer node to activate its two-way audio client to call into a conference call or call.

In aspects, the technology disclosed relates to a method in a system including a backend system, a network comprising at least three network nodes and at least one wearable device having short-range wireless communication capabilities and comprising a microphone and at least one of at least one sensor and a user input means, the method comprising:
a. assigning one of the at least one of the network nodes to be managing conference calls and/or calls for a wearable device;
b. transmitting, by the backend system, downlink data to the network;
c. receiving, by the hub node having the gatherer node role for a wearable device, the downlink data from the backend system; and
d. initiating, by the hub node having the gatherer node role for the wearable device and based on or in response to the downlink data from the backend system is indicating to the gatherer node that the wearable device is to participate in a conference call or call, the establishment of an internal transmission channel or path within the network for routing sound recorded by the microphone of the wearable device as audio data to the node having the gatherer node role for the wearable device.

In embodiments, the above method is further comprising the transmission of audio data recorded by the microphone over the established internal transmission channel or path includes the transmission of audio data over at least one established Bluetooth link, e.g. BLE links.

In certain embodiments, the above method is further comprising the transmission of audio data over the internal transmission channel or path includes the transmission of audio data between at least one established Bluetooth communication links between network nodes and/or the established Bluetooth communication link between the connection node for the wearable device and the wearable device.

In embodiments, the above step of initiating includes initiating the establishment of an internal transmission channel or path within the network for routing both sound recorded by the microphone of the wearable device as audio data to the network node managing conference calls and/or calls for a wearable device and for routing to the network node incoming audio data of a conference call or call from the network node to another network node so that the audio data can be played out by the speaker of the other network node.

In embodiments, the above method is further comprising the transmission of audio data recorded by the microphone of the wearable device and incoming audio data to be played out by the speaker of the other network node over the established internal transmission channel or path includes the transmission of audio data over at least one established Bluetooth link, e.g. BLE links.

In certain embodiments, the above method is further comprising the transmission of audio data recorded by the microphone of the wearable device and incoming audio data to be played out by the speaker of the other network node over the established internal transmission channel or path, wherein the audio data is transmitted over at least one established Bluetooth communication link, e.g. BLE link, established between two network nodes and/or over the established Bluetooth communication link, e.g. BLE link, between a node of the network and the wearable device.

In embodiments, the above method is further comprising that the downlink data is received by one network node which manages conference calls or calls for the wearable device and thereby responsible for initiating the internal transmission channel or path and for determining which of the at least three nodes is to activate its speaker to play out audio data received from the conference call or call.

In embodiments, the above method is comprising that each of the at least one wearable device is assigned one of the network nodes to be responsible for the wearable device, and wherein the network node being responsible for a wearable device is responsible for dynamically assigning one of the network nodes, including itself, to be the network role responsible for managing conference calls or calls in which the wearable device is participating.

In embodiments, the above method is comprising that each of the at least one wearable device is assigned one of the network nodes to be responsible for the respective wearable device, and wherein the network node being responsible for a wearable device is responsible for determining and dynamically changing which only one of the nodes of the SON is currently having an established short-range wireless connection with the wearable device.

In aspects, the technology disclosed relates to a method in a system including a backend system, a self-organizing network (SON) comprising at least three network nodes including at least one hub node, at least two satellite nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. assigning one of the at least one hub node to have the role of a gatherer node for a wearable device;
b. transmitting, by the backend system, downlink data to the SON;
c. receiving, by the hub node having the gatherer node role for a wearable device, the downlink data from the backend system; and
d. initiating, by the hub node having the gatherer node role for the wearable device and in response to the downlink data from the backend system is indicating to the gatherer node that the wearable device is to participate in a conference call or call, the establishment of an internal transmission channel or path within the SON for routing sound recorded by the microphone of the wearable device as audio data to the node having the gatherer node role for the wearable device.

In aspects, the technology disclosed relates to a method in a system including a backend system, a self-organizing network (SON) comprising at least three network nodes including at least one hub node, at least two satellite nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. assigning, by the SON, one of the at least one hub node to have the role of a controller node for the wearable device;
b. assigning, by the controller node for the wearable device, one of the at least one hub node to have the role of a gatherer node for a wearable device;
c. transmitting, by the backend system, downlink data to the SON;
d. receiving, by the hub node having the gatherer node role for a wearable device, the downlink data from the backend system; and
e. initiating, by the hub node having the gatherer node role for the wearable device and in response to the downlink data from the backend system is indicating to the gatherer node that the wearable device is to participate in a conference call or call, the establishment of an internal transmission channel or path within the SON both for routing sound recorded by the microphone of the wearable device as audio data to the node having the gatherer node role for the wearable device and for routing to the gatherer node incoming audio data of a conference call or call from the gatherer node to one of the other network nodes so that the audio data can be emitted through the speaker of the other network node.

In embodiments, the method of the technology disclosed comprises that the initiation of the internal transmission channel or path by the gatherer node for the wearable device is triggered in response to the received data is indicating at least one of an alert state, a changed physical condition, a changed health state, a changed physical orientation of the person wearing the wearable device and that a call should be established with the wearable device.

In embodiments, the method of the technology disclosed further comprises:
forwarding, by the hub node having the gatherer node role for the wearable device, audio data recorded by the microphone of the wearable device and transmitted over the internal transmission channel or path within the SON to at least one of an external system for managing the conference call or call in which the wearable device and a mobile communication device associated with the wearable device is participating and a mobile communication device associated with the wearable device.

In embodiments, the method of the technology disclosed further comprising:
transmitting, by the hub node currently having the gatherer node role for said wearable device and in response to the downlink data is indicating that a call should be established with the wearable device, control data adapted for instructing the activating of the microphone of the wearable device.

In embodiments, the method of the technology disclosed further comprises:
determining, by the hub node having the gatherer node role for the wearable device and in response to the downlink data is indicating that a call should be established with the wearable device, which of the at least three nodes is to activate its speaker and emit audio data received from the conference call or call routed over the internal transmission channel or path.

In embodiments, the determining by the gatherer node of which of the at least three nodes is to activate its speaker and emit audio data received from the conference call or call via the internal transmission channel or path is at least partly based on at least one of signal strength values of short-range wireless signals transmitted by the wearable device and which are measured by at least one of the network nodes and data the wearable device which is collected by at least one other hub node and then shared with the other hub nodes by distribution within the SON.

In embodiments, the method of the technology disclosed further comprises:
receiving, by the backend system and from the SON, e.g. the controller node for the wearable device, at least one of input data obtained by the user input means of the wearable device and sensor data obtained by at least one sensor of the wearable device; and
transmitting, by the backend system and at least partly based on the received input data and/or sensor data obtained by the user input means and/or at least one sensor of the wearable device, the downlink data to the SON.

In embodiments, the at least one of input data and sensor data obtained by the wearable device is transmitted to the backend system by the one hub node assigned by the SON to have the controller node role for the wearable device, wherein the controller node is responsible for determining and dynamically changing which of the nodes of the SON is currently having an established short-range wireless connection with the wearable device.

In embodiments, the method of the technology disclosed comprises that the downlink data is received by the one hub node assigned by the SON to have the gatherer node role for the wearable device and thereby responsible for initiating the internal transmission channel or path and for determining which of the at least three nodes is to activate its speaker to emit or play audio data received from the conference call or call via the internal transmission channel or path.

In embodiments, the method of the technology disclosed comprises that each of the at least one wearable device is assigned one of the hub nodes to have a controller node role for the wearable device, and wherein the hub node having the controller node role for a wearable device is responsible for dynamically assigning one of the network nodes to have the gatherer node role for the wearable device it is responsible for.

In embodiments, the downlink data is received by the one hub node assigned by the SON to have the controller node role for the wearable device and responsible for determining and dynamically changing which only one of the nodes of the SON is currently having an established short-range wireless connection with the wearable device, and wherein the controller node is configured to share the received downlink data with the hub node having the gatherer node role for the wearable device.

In embodiments, the method includes that each of the at least one wearable device is assigned only one of the at least one hub node to have the controller node role for the wearable device and be configured for dynamically determining and changing which node is currently having a short-range wireless communication link established with the wearable device.

In embodiments, each of the at least one wearable device is assigned only one of the at least one hub node to have the controller node role for the wearable device and be configured for dynamically determining and changing which only one node is currently having the gatherer node role for the wearable device(s) it is responsible for.

In embodiments, the method of the technology disclosed further comprises:
initiating, by the backend system and in response to uplink data received from the SON is indicating to the backend system that a conference call or call should be established with one of the at least one wearable device, a conference call or call in which the wearable device and a mobile communication device linked to the wearable device in the backend system are to participate.

In embodiments, the method of the technology disclosed further comprises initiating, by the backend system, the establishment of a conference call or call by transmitting a request to a third-party call facilitator requesting the third-party call facilitator to start a conference call or call in which the wearable device and a mobile communication device linked to the wearable device in the backend system can call into.

In embodiments, the method of the technology disclosed further comprises initiating, by the backend system, the establishment of a conference call or call by transmitting a request to the hub node having the gatherer node role for a wearable device requesting the gatherer node to activate its two-way audio client to call into the conference call or call.

In embodiments, the controller node for the respective wearable device is configured to determine a specific alert state and/or a health state for the individual wearing the personal device at least partly based on received sensor data and/or input data associated with the personal device.

In embodiments, the controller node for a wearable device may be configured to transmit alert state data and/or alarm data to a local or remote processing arrangement, e.g. backend system, based on sensor data and/or input data received from the wearable device. The local or remote processing arrangement may then be configured to determine that a conference call or call should be established with the wearable device by informing and/or transmitting a message or request to the wearable device and a mobile communication device linked to the wearable device, e.g. that belongs to a relative or the staff members at the facility.

In embodiments, each of the network nodes of the node network is configured to first measure signal strengths of short-range wireless broadcast signals received from a wearable device and then automatically transmit or forward data indicating the measured signal strengths to the identified controller node responsible for the personal device transmitting the short-range wireless broadcast signals.

In embodiments, the controller node currently assigned to a wearable device may be configured to determine which of the network nodes including itself is to be the node having a gatherer node role for the wearable device at least partly based on the received data indicating the measured signal strengths.

In embodiments, the controller node for the respective personal device may be configured to set an alert state or alarm data based on received sensor data and/or input data and may also be configured to determine a position and/or a location for their respective wearable device at least partly based on the received data indicating the measured signal strengths. The decision by the backend system or SON/gatherer node to initiate the establishment an internal transmission channel or path for connecting the wearable device to a conference call or call may then be at least partly based on the alert state or alarm data set by the controller node, and/or the decision to initiate the establishment an internal transmission channel or path may be at least partly based on the determined position and/or location for the wearable device.

In embodiments, the controller node for the respective personal device is configured to set or change a health state for the individual wearing a personal device at least partly based on received sensor data obtained by and/or originating from at least one medical sensor of the wearable device.

In embodiments, the determining of which of the network nodes is to play out the audio data from the conference call or call determined by the gatherer node may be at least partly based on the received, by the controller node, signal strength indication (RSSI) of short-range wireless broadcast signals transmitted by the wearable device and which are received by the network nodes within short-range communication range with the wearable device. The network nodes within short-range communication range then measure the RSSI of short-range wireless broadcast signals and forwards the RSSI to the assigned controller node for the wearable device. The measured signal strength values may then be forwarded to the backend system which may use machine learning algorithms for processing the data and the determining by the gatherer node may be at least partly based on the processed signal strength values. The measured RSSI values of short-range wireless broadcast signals transmitted by a wearable device may thus be used by the network node assigned to the wearable device for determining which of the network nodes, including itself, should receive the audio data over the internal transmission channel or path and play out the audio data through its speaker. In embodiments, the controller node may then transmit data to the gatherer node (assigned by the controller) indicating which of the network node is playing out audio data from the conference call or call.

In aspects, the technology disclosed relates to a method in a system including a network comprising network nodes and a wearable device with short-range wireless communication capabilities, said method comprising:
a. assigning a network node to be controller node for the personal device;
b. assigning, by said controller node, a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data associated with said personal device;
f. forwarding, from the assigned connection node to the controller node for said personal device, said message or data to said assigned controller node for said personal device; and
g. transmitting, from said personal device, a short-range wireless broadcast signal, wherein said short-range wireless broadcast signal contains data associated with said personal device and is transmitted while said short-range wireless communication link between said connection node and said personal device is active, and wherein said short-range wireless broadcast signal is received or detected by any network node currently within short-range communication range with said personal device; and
h. forwarding, by any and all network nodes currently within short-range communication range with said personal device and to said controller node for said personal device, at least one of data extracted from said short-range wireless broadcast signal and measured, by each of the network nodes received the broadcast signal, respective signal strengths of the received short-range wireless broadcast signal.

In embodiments, said at least one of status data, sensor data and event data associated with said personal device is provided, e.g. automatically provided, to said controller node by forwarding said received message from said connection node or by transmitting, from said connection node, a message including data corresponding to, or at least partly based on, said received at least one of status data, sensor data and event data associated with said personal device.

In embodiments, the message from transmitted from the connection node is containing sensor data and/or event data, and the method is further comprising:
a. determining, by the controller node and at least partly based on received sensor data and input data associated with the wearable device, a specific alert state and/or a health state for the individual wearing the personal device.

In embodiments, the message transmitted from the connection node is containing sensor data and/or event data, and the method is further comprising:
b. transmitting, from the controller node to a local or remote processing arrangement, at least one of said sensor data and/or input data indicating a changed health state and/or alert state for the individual wearing the personal device.

In embodiments, the message transmitted from the connection node is containing sensor data and/or input data, and the method is further comprising:
c. transmitting, from the controller node, the sensor data and/or event data associated with the personal device to a local or remote processing arrangement; and
d. determining, by the local or remote processing arrangement, the current health state and/or alert state for the individual wearing the personal device based on said sensor data and/or event data, wherein processing of the sensor data for determining the current health state and/or alert state is performed by the local or remote processing arrangement using a trained machine learning algorithm, and wherein the changed health state and/or alert state is adapted to trigger the local or remote processing arrangement to initiate the establishment of a conference call or call in which the wearable device and a mobile communication device linked to the wearable device is to participate.

In embodiments, the method is further comprising:
e. determining, by the local or remote processing arrangement, a changed health state and/or alert state for the individual wearing the personal device controller node, wherein the determining is at least partly based on said received sensor data and event data associated with the personal device is indicating a specific alert state and/or a health state for the individual wearing the personal device; and
f. transmitting, from the controller node to a local or remote processing arrangement, data indicating the determined changed health state and/or alert state for the individual wearing the personal device, and wherein the changed health state and/or alert state is adapted to trigger the local or remote processing arrangement to initiate the establishment of a conference call or call in which the wearable device and a mobile communication device linked to the wearable device is to participate.

In embodiments, the method is further comprising:
g. setting, by the local or remote processing arrangement, an alarm or alert state for the personal device and/or individual wearing the personal device, wherein said setting of an alarm is at least partly based on the determining of a changed health state and/or alert state for the individual wearing the personal device, and wherein the alarm or alert state triggers the local or remote processing arrangement to initiate the establishment of a conference call or call in which the wearable device and a mobile communication device linked to the wearable device is to participate.

In embodiments, the technology disclosed provides a solution for fast response to obtained sensor data or an identified event, e.g. health sensor data indicating a health state, e.g. a deteriorated health condition, of the individual wearing the personal device or the event of pressing an alarm button on a personal device such as a wristband. The fast response solution according to the technology disclosed is achieved by the transmission of sensor data and/or event/input data indicating health sensor data, e.g. obtained by a medical sensor, or the occurrence of an event such as the pressing an alarm button on the personal device or activating an voice activation means connected to the microphone of the personal device, both via an established short-range wireless connection with one of the network nodes and via short-range wireless broadcast transmissions, e.g. via Bluetooth, from the personal device and which are received by those network nodes which are within short-range wireless communication range, e.g. Bluetooth range, with the respective personal device transmitting the broadcast signals.

In embodiments, the technology disclosed provides a solution for dynamically and accurately determining the indoor position or location for a personal device by measuring, by each of the network nodes which are within short-range communication range with the personal device, the

In certain embodiments, the current position or location, e.g. room, for the personal device may at least partly be determined by a trained machine learning algorithm used by the controller node or a locally-installed or remote processing arrangement such as a backend system communicatively coupled to the controller node, since machine learning is an efficient way of evaluating complex signals in situations where large amounts of data is collected. The trained machine learning algorithm may then use at least the signal strength received from the nodes within short-range wireless communication range (including the current controller node and the current connection node for the personal device if any of these two nodes are within short-range wireless communication range with the personal device to receive the broadcasts) and/or sensor data detected by a movement sensor of the personal device to determine the current position or location for the personal device

In aspects, the technology disclosed relates to a method for a system comprising at least two network nodes configured to share data with the other nodes via established communication links between the nodes, e.g. an IP-based network or BLE links, and a plurality of personal devices with Bluetooth communication capabilities, said method comprising:
a. assigning, e.g. by the nodes of the system using a common decision model which is known to all of the at least two network nodes, only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. assigning, by said assigned only one single controller node, only one single gatherer node for said personal device, wherein said gatherer node is sole responsible for calling into conference calls and calls in which the personal device is participating and routing audio data of the conference call or call to and from said personal device;
d. establishing, by said assigned only one single connection node, a Bluetooth connection between said only one single connection node and said personal device, wherein said Bluetooth connection is the only currently established Bluetooth connection from the network to said personal device;
e. transmitting, from said personal device, data via said established Bluetooth connection to said only one single connection node; and
f. transmitting, from said personal device, Bluetooth broadcast signals, wherein said Bluetooth broadcast signals are transmitted while said personal device has said established Bluetooth connection with said only one single connection node.

### BRIEF DESCRIPTION OF DRAWINGS

Preferred embodiments of a system according to the technology disclosed will be described more in detail below with reference to the accompanying drawings wherein: FIG. 1 schematically shows an example embodiment of a system for monitoring an individual within predetermined facilities, in accordance with one or more embodiments described herein.

FIG. 2 is a schematic illustration of an example embodiment of a system for monitoring an individual within predetermined facilities, in accordance with one or more embodiments described herein.

FIG. 3 is a schematic illustration of an example embodiment of a system for monitoring an individual within predetermined facilities, in accordance with one or more embodiments described herein.

FIG. 4 schematically illustrates a method for monitoring an individual within predetermined facilities, in accordance with one or more embodiments described herein.

FIG. 5 schematically illustrates a method for monitoring an individual within predetermined facilities, in accordance with one or more embodiments described herein.

FIG. 6 schematically illustrates a method for monitoring an individual within predetermined facilities, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In the drawings, similar details are denoted with the same reference number throughout the different embodiments. In the various embodiments of the system, e.g. monitoring system, according to the technology disclosed, the different subsystems are denoted. The “boxes”/subsystems shown in the drawings are by way of example only and can within the scope of the technology disclosed be arranged in any other way or combination.

In aspects, the technology disclosed relates to the assignment of a gatherer node for handling conference calls or calls and establishing an internal transmission channel or path which is adapted to be used for connecting a wearable device to the gatherer node with the purpose of transmitting audio data obtained by a microphone of the wearable device to the conference call or call and for transmitting audio data from the conference call or call to a network node which is determined to be relatively close to the wearable device, e.g. satellite node, for play out so that the individual/patient carrying the wearable device can hear the audio data played out by the network node.

In embodiments, the technology disclosed relates to methods, a system comprising a self-organizing network (SON) of nodes and at least one wearable device each configured to transmit data associated with the respective wearable device both via an established short-range wireless connection links with one of the network nodes of the node network and via short-range wireless broadcasts which may be received by any of the network nodes that are within short-range wireless communication range with the wearable device.

The technology disclosed also relates to a system and methods for transmitting data associated with a personal device worn by an individual both via an established short-range wireless connection link between the personal device and a network node and via short-range wireless broadcasts from the personal device.

In aspects, the wearable device is adapted to be worn by an individual and is configured to transmit short-range wireless broadcast signals while a short-range wireless connection between the wearable device and a node of the network of nodes is active.

In aspects and embodiments, the technology disclosed relates to a system for monitoring individuals including a backend system and a self-organizing network (SON) comprising:
at least three network nodes including at least two network nodes comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means, wherein each of the at least one wearable device is configured to establish a short-range wireless communication link with one of the network nodes and each of the at least two network nodes is configured to, in response to receiving downlink data from the backend system indicating that a conference call or call should be established with the wearable device, initiate the establishment of an internal transmission channel or path within the SON for routing sound recorded by the microphone of the wearable device as audio data to the network node and for routing to the gatherer node incoming audio data of a conference call or call from the network node to another network node so that the audio data can be played out by the speaker of the other network node.

In aspects and embodiments, the technology disclosed relates to a system for monitoring individuals including a backend system and a self-organizing network (SON) comprising:
at least three network nodes including at least two network nodes comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means, wherein a wearable device is configured to establish a short-range wireless communication link with one of the network nodes and each of the at least three network nodes is configured to, in response to receiving downlink data from the backend system indicating that a conference call or call should be established with the wearable device, initiate the establishment of an internal transmission channel or path within the SON for routing sound recorded by the microphone of the wearable device as audio data to the network node and for routing to the gatherer node incoming audio data of a conference call or call from the network node to another network node so that the audio data can be played out by the speaker of the other network node.

In embodiments, the backend system is configured to transmit the downlink data to a network node of the SON in response to receiving uplink data from the SON indicating to the backend system at least one of an alert state, a changed physical condition, a changed health state, a changed physical orientation of the person wearing the wearable device and that a call should be established with the wearable device.

In embodiments, the downlink data received by the network node is included in at least one of a configuration file which is continuously updated by the backend system and transmitted to the SON, a call initiation request including data for identifying a wearable device which is to participate in a conference call or call, and control data indicating to the network node assigned to the wearable device that the wearable device is to participate in a conference call or call.

In embodiments, the backend system is configured to continuously update data associated with the wearable device based on input data and/or sensor data received by the backend system from the SON, and wherein the downlink data which may trigger the initiation of an internal transmission channel or path within the SON is included in a configuration file continuously updated and transmitted by the backend system to the network node of the SON.

In embodiments, the network node is configured to initiate the establishment of an internal transmission channel or path within the SON for the purpose of establishing a conference call or call in which the wearable device is to participate in response to the updated and transmitted configuration file is indicating to the network node at least one of an alert state, a changed physical condition, a changed health state, a changed physical orientation of the person wearing the wearable device and that a call should be established with the wearable device.

In embodiments, the network node is configured to initiate the establishment of an internal transmission channel or path to a network node having an established short-range wireless communication link with the wearable device where the internal transmission channel or path includes at least one communication link between the network node and another network node. In certain embodiments, the network node is configured to use the same internal routing table for initiating the establishment of the internal transmission channel or path for routing audio data obtained by the microphone of the identified wearable device from the other network node to the network node as for the routing of incoming audio data of a conference call or call to the SON from the network node to the other network node for play out by the speaker of the other network node.

In aspects, the technology disclosed relates to a method for monitoring individuals in a system including a backend system, a self-organizing network (SON) comprising at least three network nodes including at least two network nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. transmitting, by the backend system, downlink data to the SON;
b. receiving, by a network nodes, said downlink data from the backend system; and
c. initiating, by the network node and in response to the downlink data from the backend system is indicating to said network node that the wearable device is to participate in a conference call or call, the establishment of an internal transmission channel or path within the SON for routing sound recorded by the microphone of the wearable device as audio data to the network node assigned to the wearable device and for routing to the network node incoming audio data of a conference call or call from the network node to one of the other network nodes so that the audio data can be played out by the speaker of the other network node.

In embodiments, the method is further comprising:
forwarding, by said network node, audio data recorded by the microphone of the wearable device and transmitted over the internal transmission channel or path within the SON to at least one of an external system for managing the conference call or call in which the wearable device and a mobile communication device associated with the wearable device is participating and a mobile communication device associated with the wearable device.

In embodiments, the method is further comprising:
transmitting, by the network node and in response to the downlink data is indicating that a call should be established with the wearable device, control data for activating the microphone of the wearable device.

In embodiments, the method is further comprising:
determining, by the network node and based on or in response to the downlink data is indicating that a call should be established with the wearable device, which of the at least three nodes is to activate its speaker to play out audio data received from the conference call or call routed over the internal transmission channel or path.

In embodiments, the determining by the network node which of the network nodes is to activate its speaker to play out audio data received from the conference call or call is at least partly based on at least one of signal strength values of short-range wireless signals transmitted by the wearable device and which are measured by at least one of the network nodes, and data the wearable device which is collected by at least one other network node and then shared with the other hub nodes by distribution within the SON.

In embodiments, the method is further comprising:
receiving, by the backend system and from the SON, at least one of input data obtained by the user input means of the wearable device and sensor data obtained by at least one sensor of the wearable device; and
transmitting, by the backend system and at least partly based on the received input data and/or sensor data obtained by the user input means and/or at least one sensor of the wearable device, the downlink data to the SON.

In embodiments, the at least one of input data and sensor data obtained by the wearable device is transmitted to the backend system by at least one of the network nodes, wherein said at least one network node is responsible for determining and dynamically changing which of the nodes of the SON is currently having an established short-range wireless connection with said wearable device.

In embodiments, the method is further comprising:
initiating, by the backend system and in response to uplink data received from the SON is indicating to the backend system that a conference call or call should be established with the wearable device, a conference call or call in which the wearable device and a mobile communication device linked to the wearable device in the backend system are to participate.

In embodiments, the method is further comprising initiating, by the backend system, the establishment of a conference call or call by transmitting a request to a third-party call facilitator requesting the third-party call facilitator to start a conference call or call in which the wearable device and a mobile communication device linked to the wearable device in the backend system can call into.

In embodiments, the method is further comprising initiating, by the backend system, the establishment of a conference call or call by transmitting a request to the network node assigned to the wearable device requesting the network node to activate its two-way audio client to call into the conference call or call.

In aspects, technology disclosed relates to methods and a system comprising at least two network nodes and at least one personal devices, or wearable devices, each configured to transmit at least one short-range wireless broadcast signal which is received by any of the at least two network nodes within short-range wireless communication range with the respective personal device. The transmission of the at least one short-range wireless broadcast signal is performed while an established communication connection or link between the wearable device and one of the network nodes which is the assigned connection node for the respective personal device is active. The transmission of broadcast signals may be performed in parallel with or while the personal device is transmitting messages via the established short-range wireless communication link.

In aspects, technology disclosed relates to methods and a system comprising at least three network nodes and a plurality of personal devices, or wearable devices, each configured to continuously, e.g. periodically, transmit short-range wireless broadcast signals which are received by any of the at least three network nodes within short-range wireless communication range with the respective personal device. The transmissions of the short-range wireless broadcast signals are performed while an established communication link between the personal device and one of the network nodes which is the assigned connection node for the respective personal device is active. The transmission of the short-range wireless broadcast signals, e.g. Bluetooth/BLE signals, may be performed in parallel with or while the personal device is transmitting messages via the established short-range wireless connection or communication link, e.g. Bluetooth/BLE communication link.

In embodiments, the personal devices are adapted to be worn by individuals and are configured to transmit short-range wireless broadcast signals while a short-range wireless connection between the personal device and a node of the network of nodes is active. The hub node assigned to have the controller node role for a wearable device is configured to change which of the network nodes is having a short-range wireless communication link the position or location of the individual and take actions on behalf of the individual wearing the personal device and in response to received data contained in broadcast signals forwarded by other network nodes, including other hub nodes and satellite nodes, and/or status data, sensor data and/or event data received and forwarded to the controller node by the current connection node having a short-range wireless communication link with the wearable device.

According to aspects and certain embodiments of the technology disclosed, data or information contained in short-range wireless broadcast message received by the connection node, e.g. a satellite node, via the established short-range wireless communication link is forwarded, via short-range wireless communication links established between the network nodes (e.g. hub nodes), to be obtained by the assigned controller node for the wearable device. The controller node, which has sole responsibility for the wearable device, may be further configured to use input data and/or sensor data obtained by the wearable device which is forwarded from the currently assigned connection node and/or input data and/or sensor data obtained by the wearable device which is received from the other nodes to take actions or make decisions on behalf of the wearable device, e.g. to change gatherer node or connection node for the wearable device at least partly based on the received and obtained input data and/or sensor data.

According to aspects and certain embodiments of the technology disclosed, data or information contained in short-range wireless broadcast message received by the connection node, e.g. a satellite node, via the established short-range wireless communication link is forwarded via an IP-based network to the assigned controller node for the wearable device. The controller node, which has sole responsibility for the personal device, may be further configured to use input data and/or sensor data obtained by the wearable device which is forwarded from the currently assigned connection node and/or input data and/or sensor data obtained by the wearable device which is received from the other nodes to take actions or make decisions on behalf of the wearable device, e.g. to change gatherer node or connection node for the wearable device at least partly based on the received and obtained input data and/or sensor data.

In aspects, the technology disclosed relates to a system including a network comprising at least two network nodes and a plurality of personal devices with short-range wireless communication capabilities. The system is configured to assign a connection node among the at least two network nodes to each of the plurality of personal devices. The connection node is responsible for establishing a short-range wireless communication link with the personal device it is assigned connection node for.

In aspects, the technology disclosed relates to a system including a network comprising at least three network nodes and a plurality of personal devices with short-range wireless communication capabilities. The system is configured to assign a connection node among the at least three network nodes to each of the plurality of personal devices. The connection node is responsible for establishing a short-range wireless communication link with the personal device it is assigned connection node for.

According to the technology disclosed, each of the plurality of personal devices is configured to transmit short-range wireless broadcast signals while their respective established short-range wireless connection with their respectively assigned connection node is active.

In embodiments, the system is configured to assign a controller node for the respective of the plurality of personal device, and wherein each controller node for the respective personal device is configured to assign a connection node for the personal device and to receive, from the other network nodes, status data, sensor data and/or event data associated with the personal device it is responsible for.

In embodiments, the IP-based network connecting the hub nodes is a separate network and is configured to use a different communication protocol from the communication protocol used for establishing the short-range wireless communication links between the at least one wearable device and the network nodes, e.g. satellite nodes. The short-range wireless communication links for connecting the wearable devices to one of the network nodes, either a satellite node or a hub node, may be Bluetooth communication links, e.g. BLE links.

In embodiments, the hub nodes are configured to share data with the other nodes via an IP-based network connecting the hub nodes, e.g. all of the hub nodes of the SON may be wirelessly connected via WiFi connections or via Ethernet connections, thereby connecting the hub nodes so that they continuously can share their obtained input data and/or sensor data with the other hub nodes via distribution between the hub nodes.

In embodiments, the hub nodes are configured to share data with the other nodes via established short-range wireless communication link, e.g. Bluetooth/BLE links, thereby connecting the hub nodes so that they continuously can share their obtained input data and/or sensor data with the other hub nodes via distribution between the hub nodes.

In embodiments, the at least one sensor of the wearable device may be a health or medical sensor such a body temperature sensor for determining the body temperature of the wearer, or may include a blood pressure sensor, a heart rate sensor, a skin galvanometric level sensor etc.

In embodiments, the personal device may be a wristband and at least one sensor of the personal device may include at least one health or medical sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a temperature sensor for obtaining sensor data for determining the body temperature of the wearer, or may include a blood pressure sensor, a heart rate sensor, a skin galvanometric level sensor etc.

In aspects, the technology disclosed relates to a system including a network comprising at least three network nodes configured to share data with the other nodes via established link of an IP-based network or BLE links and a plurality of personal devices with Bluetooth communication capabilities, said plurality of personal devices are each assigned only one controller node and only one gatherer node among the at least two network nodes, wherein the only one controller node is responsible for assigning only one connection node among the at least three network nodes to be responsible for establishing a Bluetooth connection with the personal device the controller node is responsible for, and wherein said plurality of personal devices are each configured for transmitting Bluetooth broadcast signals while having an established Bluetooth connection with their respectively assigned only one connection node.

In embodiments, the only one controller node assigned to the respective personal device is configured to assign and dynamically change which of the at least two network nodes is currently the only one gatherer node for their respective personal devices at least partly based on at least one of contents and received signal characteristics of Bluetooth broadcast signals transmitted from the personal device and which are received by a plurality of other nodes than the controller node.

In embodiments, each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, said system comprises at least one mobile communication device and a backend system and network configured to receive alarm data indicating the occurred trigger event from the only one controller node responsible for the personal device which determined that the trigger event occurred, and wherein said backend system and network is further configured to transmit a call initiation request both to the personal device and to at least one mobile communication device in response to receiving said alarm data from the only one controller node. The call initiation request may include a calling number and/or SIP credentials so that the personal device and the mobile communication device can call into the conference call or call.

In embodiments, said personal device is a wristband and said trigger event or input data triggering the establishment of a conference call or call may be the action of pressing a button on the wristband or activating a voice activation means of the personal device.

In embodiments, the assigned controller node for a personal device is responsible for dynamically assigning only one gatherer node and one connection node for the personal device at a time. The controller node assigned to a personal device may be assigned according to a common decision model known to and used by the at least two network nodes and following the sharing or distribution of data associated with the personal device. In embodiments, the data shared between the nodes may then be used as input values to the common decision model for dynamically changing controller node for the personal device. The controller node is typically always responsible for dynamically assigning both one node to be connection node and one node to be the gatherer node for the personal device.

According to aspects of the technology disclosed, there is only one connection node for establishing a short-range wireless connection with the personal device and the system is configured so that the controller node for the personal device has the sole responsibility for changing gatherer node and connection node for the personal devices. This provides for a fast response yet robust and scalable solution for monitoring personal devices, e.g. wristbands, necklaces or clips carried or worn by individuals moving between locations or rooms of a facility such as patients in a nursing home. The established short-range wireless connection is then the only connection between the network and the personal device. According to this embodiment, the personal device may then still transmit short-range wireless broadcast signals to provide the other network nodes within short-range wireless communication range with status data, obtained sensor data, event data and/or input data associated with the personal device.

In certain embodiments, the technology disclosed relates to methods and a system for monitoring an individual within predetermined facilities by identifying at least one of an alert state, a changed physical condition, a changed health state, a changed physical orientation of the person wearing the wearable device, e.g. with the purpose of determining whether a call should be established with the wearable device. The system may comprise at least three network nodes and a plurality of personal devices each carried or worn by the respective monitored individual, where each of the plurality of personal devices comprises at least one sensor and having short-range communication capability, e.g. Bluetooth communication capability, with which the respective personal device is arranged to communicate with the network nodes. The personal device may also comprise a storage means and at least one processing device.

In embodiments, the personal device may be a wristband and the at least one sensor of the personal device may comprise at least one medical sensor, or health sensor, adapted for obtaining sensor data for determining the medical and/or physical condition of the wearer of the wristband, e.g. a medical sensor configured to sense a characteristic of the user and generate a sensor output signal.

In embodiments, the personal device may be a wristband and the at least one sensor of the personal device may comprise at least one health sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a body temperature sensor for obtaining sensor data for determining the body temperature of the wearer, or may include a blood pressure sensor, a heart rate sensor, a skin galvanometric level sensor etc.

The wearable devices may broadcast their own universal unique identifiers (UUIDs) and seek to establish connections with Bluetooth devices within its Bluetooth communication range. The connection node assigned, by the controller node, to a certain wearable device may then establish a Bluetooth connection with the wearable device by transmitting a connection request via Bluetooth to the wearable device where the connection request may include the unique identifier (UUID) of the wearable device.

According to aspects, the network nodes may be self-organizing in that the assignment of the one controller node having the sole responsibility for a specific personal device is a distributed decision which is preceded by at least one of distributing and sharing data between the network nodes and by using a predetermined and common decision model with dynamic input values/parameters which are changed with the data distributed and shared between the network nodes.

According to embodiments, the network nodes may be self-organizing in that the decision to determine or change controller node for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to embodiments of the technology disclosed, personal devices transmits data to the nodes in the node network using Bluetooth, e.g. via Bluetooth broadcasts and an established Bluetooth connection with one of the network nodes, and the transmission of data, e.g. data associated with the individual personal device, e.g. data received from a certain personal device or associated with a certain personal device, may be shared between network nodes via established links, e.g. Bluetooth links or IP-based links, connecting the network nodes. In embodiments, the established short-range wireless communication link between the connection node and the personal device is a bidirectional short-range wireless communication link.

In embodiments, the technology disclosed provides a fast response yet robust solution for acting on an identified event by parallel and/or redundant data transmission of event data representing or reflecting the event via both an established short-range wireless connection with one of the nodes and via short-range wireless broadcast messages from the personal device to the nodes which are within short-range wireless communication range.

In embodiments, the technology disclosed provides a solution for dynamically and accurately determining the indoor position or location for a wearable device without any use of high energy-consuming GPS in the personal devices or beacons, by measuring, by each of the network nodes which are within short-range communication range with the personal device, the signal strength of short-range wireless broadcast signals, e.g. Bluetooth broadcast signals, received from the wearable device.

In embodiments, the controller node for the wearable device may then receive, via the IP-based network or BLE links between the network nodes, data and/or information representing or reflecting the measured signal strengths from each of the plurality of other nodes within short-range communication range with the personal device and, based on the data and/or information, determine the current position or location for the personal device. The network node, e.g. gatherer node, responsible for establishing the internal transmission channel or path may then determine, at least partly based on the position or location (e.g. determined by the controller node), which of the network nodes, e.g. satellite nodes, to establish the internal transmission channel or path with and that should receive and play out audio data from the conference call or call.

In various embodiments, the controller node may forward the obtained data and/or information representing or reflecting the measured signal strengths from each of the plurality of other network nodes, and/or other movement sensor data obtained, to a backend network which determines the current position or location for the wearable device.

In various embodiments, the controller may in addition use other obtained sensor data, e.g. sensor data from the movement sensor of the wearable device (e.g. an accelerometer), to determine and/or calculate the current position and/or location for the personal device.

In certain embodiments, the current position or location for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically changing the connection node for a personal device, by measuring, by each of the network nodes which are within short-range communication range with the personal device, the signal strength of short-range wireless broadcast signals, e.g. Bluetooth broadcast signals, received from the wearable device.

In embodiments, the controller node for the personal device may then receive, via the IP-based network or established Bluetooth/BLE communication links, data and/or information representing or reflecting the measured signal strengths from each of the plurality of other nodes within short-range communication range with the personal device and, based on the data and/or information, determine to change connection node for the personal device.

In various embodiments, the controller node may forward the obtained data and/or information representing or reflecting the measured signal strengths from each of the plurality of other nodes, and/or other movement sensor data obtained, to a backend network which may determine, based on the data and/or information (e.g. data processed by a machine learning algorithm used by the backend system), that the gatherer node or connection node for the personal device should be changed. The backend system may then provide the network with downlink data, e.g. control data or an updated configuration file, which indicates to a controller node for a wearable device that the gatherer node or connection node for the wearable device should be changed or that indicates which of the network nodes should play out audio data received from a conference call or call in which the wearable device is to participate.

In various embodiments, the controller node may in addition use other obtained sensor data, e.g. sensor data from a movement sensor of the personal device, e.g. an accelerometer, to determine that the connection node for the personal device should be changed.

In certain embodiments, the change of connection node for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine that the connection node for the personal device should be changed.

The systems and networks, e.g. piconets or SON, known in the art further does not disclose a system, e.g. monitoring system, comprising a node network and personal devices with broadcast capabilities which is self-organizing and scalable by dynamically assigning roles to the individual nodes, e.g. continuously and automatically assigning roles to the nodes, where each assignment of a role to a node relates to managing and control of only one of the personal devices. In particular, the systems and network solutions known in the art are silent to dynamically assigning and changing roles to the nodes on two levels, a control level where the node has the sole responsibility for a personal device and a connection level where the node is responsible for establishing a connection or link with the personal device.

The technology disclosed introduces a node network where the hub nodes which each may have the role of a controller node or gatherer node for a wearable device communicate internally via their IP addresses or via a network of established Bluetooth/BLE links always connecting the hub nodes, but where the communication between the wearable devices and one of the nodes of the network takes place via dynamically established short-range wireless communication such as Bluetooth links. This gives a better load balancing and much higher bandwidth capacity for the communication between the wearable devices and the nodes. This also enables the node network to be more scalable and distributed, since communication between the network nodes can take place via an established network of communication links connecting all of the hub nodes to each other.

The system, e.g. monitoring system, comprising the node network of the technology disclosed is highly scalable and provides for safe and fast responses to events, obtained sensor data and the movements of the individuals, yet is robust in that each of the personal devices always has a reliable short-range wireless connection to the node network which is not disturbed or compete for short-range communication bandwidth with other devices connected to the same node of the node network.

In aspects, the technology disclosed allows for faster and more accurate decision-making and improved load balancing within a network by providing methods and a system comprising wearable devices and a network of nodes for dynamically assigning one controller node and one connection node for each personal device. In embodiments, the controller node for a personal device is dynamically assigned or changed based on a common decision model known to the network nodes and which may contain input values or parameters. The input values or parameters may be changed with the data distributed and shared between the network nodes. In certain embodiments of the technology disclosed, the consensus model as such with its input values or parameters may also be dynamically adjusted and changed over time. The gatherer node role for a wearable device is dynamically changed by the node having the controller node role for the wearable device, e.g. based on position and/or location data or signal strength values obtained or received by the controller node which are measured by itself and the other nodes of the network including the satellite nodes.

The faster and more accurate decision-making is at least partly achieved by the method of assigning, for each wearable device, one controller node with sole responsibility for the wearable device, where the controller node is provided with all data or information associated with the personal device the controller node is responsible for and which is received, obtained and/or measured by the network nodes.

The faster and more accurate decision-making may be further achieved by the controller node making all decisions for the personal device, e.g. the decision to set an alert or transmit alarm data, the assignment of connection node with which the personal device is establishing a short-range wireless communication link such as a Bluetooth connection, or the assignment of a gatherer node for handling conference calls or calls and establishing an internal transmission channel or path used for connecting the wearable device to the conference call or call. The connection node may then have an established connection with the personal device as long as the controller node considers it appropriate, e.g. as long as the signal strength measured by the connection node is above a certain threshold value and/or a signal strength measured by the connection node is higher than signal strengths measured by the other nodes of the network.

Improved load-balancing is achieved by having the controller node with sole responsibility for a personal device dynamically assigning and changing connection node for the personal device at least partly based on load-balancing factors such as the current number of established short-range wireless connections for a node, e.g. as compared to the number of connections for other nodes, to thereby distribute the amount of short-range wireless data transmitted locally.

Benefits of the technology disclosed include that the system, self-organizing network and methods provide each of the personal devices with improved short-range wireless connections by continuously and dynamically changing which of the network nodes is the sole gatherer node for a wearable device responsible for handling audio data of conference calls and calls in which the wearable device is participating and establishing of internal transmission channels or paths for connecting the wearable device to the conference calls or calls. The determining of which network node is to play out the audio data from the conference call or call through its speaker may then be at least partly based on measured signal strengths for the currently established short-range wireless connection with the connection node and short-range signal strengths values of short-range wireless broadcast signals transmitted by the wearable device and which are measured by the individual nodes of the network within short-range wireless communication range with the wearable device.

Further benefits of the technology disclosed include that the controller node is provided with all data associated with the wearable device it is responsible for in that all data or information associated with the personal device, or data or information representing or reflecting the obtained data or information, is forwarded from the other network nodes to the controller node, including data broadcasted by the wearable device and data received or measured by the currently assigned connection node for the wearable device.

According to aspects, the hub node having the gatherer node role for a wearable device is configured to initiate the establishment of an internal transmission channel or path within the SON. The internal transmission channel or path may be initiated by the gatherer node in response to the gatherer node obtaining data indicating to the gatherer node that the wearable device should participate in a conference call or call where externally-generated audio data is received by the gatherer node/SON. The internal transmission channel or path may be established with the purpose of routing sound data recorded by the microphone of the wearable device as audio data to the gatherer node. The gatherer node is then configured to forward the audio data originating from the wearable device to an external system managing the conference call or call, or may forward the audio data directly to a mobile communication device associated with or linked to the wearable device.

In embodiments of the technology disclosed, the at least one wearable device is assigned one hub node to have a gatherer node role for the wearable device which is configured to, in response to receiving or obtaining downlink data from the backend system indicating to the gatherer node that a call should be established with the wearable device, initiate the establishment of an internal transmission channel or path within the SON for routing sound data, including speech/voice data, recorded by the microphone of the wearable device as audio data to the gatherer node. In embodiments, the SON-internal transmission channel or path may also be used for routing to the SON incoming and externally-generated audio data of a conference call or call from the gatherer node to another network node, e.g. a satellite node of the SON currently having an established short-range wireless communication link with the wearable device, so that the audio data can be played out by the speaker of the other network node, e.g. the satellite node currently having an established short-range wireless communication link with the wearable device.

In embodiments of the technology disclosed, the at least one wearable device is assigned one hub node to have a gatherer node role for the wearable device which is configured to, in response to receiving or obtaining at least one of input data and sensor data originating from the wearable device which indicates at least one of an alert state, a changed physical condition, a changed health state, a changed physical orientation of the person wearing the wearable device and that a call should be established with the wearable device, initiate the establishment of an internal transmission channel or path within the SON which is adapted both for routing sound data recorded by the microphone of the wearable device as audio data to the gatherer node and for routing to the SON incoming audio data of a conference call or call from the gatherer node to another network node, e.g. a satellite node, so that the audio data can be played out by the speaker of the satellite node. In example embodiments, the input data and/or sensor data of the wearable device may then have been collected by the hub node having a controller node role for the wearable device and then shared with the hub node assigned by the controller node to have a gatherer node role for the wearable device.

According to certain aspects of the technology disclosed, the hub node having the gatherer node role for a wearable device is responsible for managing routing of audio data obtained by a microphone of the wearable device to the gatherer node and for forwarding the audio data obtained by and originating from the microphone of the wearable device to an external third-party system for managing the conference call or call so that the audio data may be listened to by the user of a mobile communication device participating in the same conference call or call. The hub node having the gatherer node role for a wearable device is also typically also responsible for managing routing of incoming audio data of the conference call or call to a speaker of a network node so that the audio data is emitted through the speaker. In embodiments, the same internal transmission channel or path is used for both routing sound data recorded by the microphone of the wearable device as audio data to the gatherer node and for routing to the SON incoming audio data of a conference call or call from the gatherer node to another network node, e.g. another hub node or a satellite node.

In embodiments, the backend system is triggered to initiate the establishment of a conference all or call in which a wearable device communicatively connected to the SON and a mobile communication device associated with or linked to the wearable device are participating. The backend system may then send a first request to a third-party facilitator/system for managing the conference call or call, requesting the third-party facilitator/system to start a conference call or call. Following the initiation of the conference call or call, the backend system may transmit a second request to the hub node who is the gatherer node for the wearable device, requesting the gatherer node to start its audio client and call into the conference call or directly call a mobile communication device associated with the wearable device.

In embodiments, the hub node having the controller node role for a wearable device is configured to receive at least one of input data from a user input means and sensor data obtained by a sensor of the wearable devices and continuously share received input data and/or sensor data with other hub nodes of the SON so that hub nodes having the gatherer node role for the wearable device receives the input data and/or sensor data and, at least partly based on the input data and/or sensor data, at least one of transmit control data to the wearable device instructing the wearable device to activate a sensor and trigger the initiation of an internal transmission channel or path within the SON and transmit control data to the wearable device instructing said wearable device to mute its microphone.

In embodiments, the hub node having the controller node role for a wearable device is configured to, in response to receiving downlink data from the backend system which indicates at least one of an alert state, a changed physical condition, a changed health state, a changed physical orientation of the person wearing the wearable device and that a call should be established with the wearable device: transmit control data to the wearable device instructing the wearable device to activate the sensor of the wearable device.

In embodiments, the technology disclosed relates to a system including a backend system, a self-organizing network (SON) comprising:
at least three network nodes including at least one hub node,
at least two satellite nodes each comprising a speaker and configured to transmit short-range wireless broadcasts and establish a short-range wireless connection link with a wearable device, and
at least one wearable device having short-range wireless communication capabilities and comprising a microphone and at least one of at least one sensor and a user input means,
wherein each wearable device is assigned a hub node to have the role of a gatherer node for the wearable device, and wherein the hub node having the gatherer node role for the wearable device is configured to, in response to receiving downlink data originating from the backend system which indicates at least one of an alert state, a changed physical condition, a changed health state, a changed physical orientation of the person wearing the wearable device and that a call should be established with the wearable device, trigger the initiation of an internal transmission channel or path within the SON which is adapted both for routing sound recorded by the microphone of the wearable device as audio data to the gatherer node and for routing to the SON incoming audio data of a conference call or call from the gatherer node to a satellite node so that the audio data can be played out by the speaker of said satellite node.

In embodiments, the hub node having the gatherer node for a wearable device is further configured to forward the audio data recorded by the microphone and received over the internal transmission channel or path within the SON to at least one of a system managing the conference call or call in which a mobile communication device associated with the wearable device is participating and a mobile communication device associated with the wearable device which is participating in a call with the wearable device.

In embodiments, the hub node having the gatherer node role is further configured to, in response to receiving the downlink data originating from the backend system which indicates at least one of an alert state, a changed physical condition, a changed health state, a changed physical orientation of the person wearing the wearable device and that a call should be established with the wearable device, transmit both control data for activating said microphone of the wearable device and control data for activating said speaker of the satellite node.

In embodiments, the backend system is configured to receive data related to a wearable device from the SON where the received data is based on input data and/or sensor data obtained by the wearable device and, in response to the received data is indicating at least one of an alert state, a changed physical condition, a changed health state, a changed physical orientation of the person wearing the wearable device and that a call should be established with the wearable device, transmit data with call setup information both to the SON and to a mobile communication device linked to the wearable device in the backend system so that the wearable device and the mobile communication device can communicate. In embodiments, the call may then be a direct call between the wearable device/SON and the mobile communication device, e.g. a mobile phone, or a conference call in which both the wearable device connected to the SON and the mobile communication device associated with/linked to the wearable device are participating and the conference call or call may be controlled by a third-party call control system.

In embodiments, the backend system is configured to transmit the downlink data to the SON at least partly based on at least one of input data obtained by the user input means of the wearable device and sensor data obtained by at least one sensor of the wearable device and which is received by the backend system from the SON.

In embodiments, the at least one of input data and sensor data obtained by the wearable device is transmitted to the backend system by the one hub node assigned by the SON to have the controller node role for the wearable device, wherein the node having the controller node role for a wearable device is responsible for determining and dynamically changing which of the nodes of the SON is currently having the only established short-range wireless connection between the wearable device and the SON.

In embodiments, the downlink data originating from the backend system is comprised in at least one of a configuration file which is continuously updated by the backend system and transmitted from the backend system to the SON, and control data transmitted downlink from the backend system to the SON.

In embodiments, the downlink data is received by the one hub node assigned by the SON to have the controller node role for the wearable device and responsible for determining and dynamically changing which only one of the nodes of the SON is currently having an established short-range wireless connection with the wearable device, and wherein the downlink data is shared with each of the other hub nodes by distribution.

In embodiments, the hub node of the SON having the gatherer node role for a wearable device is further configured to determine which of the satellite nodes is to activate its speaker to play out audio data received from the conference call or call routed over the internal transmission channel or path at least partly based on at least one of signal strength values of short-range wireless signals transmitted by the wearable device and which are measured by at least one of the at least two satellite nodes and then received by the gatherer node via short-range wireless transmissions from the at least one of the at least two satellite nodes, and data related to the wearable device which is collected by at least one other hub node and then shared with the other hub nodes by distribution within the SON.

In embodiments, the hub node having the gatherer node role for a wearable device is further configured to determine which of the nodes is to activate its speaker to play out the incoming audio data from the conference call or call and which is routed over the internal transmission channel or path at least partly based on obtained or received data indicating which of the nodes is the only node in the SON currently having an established short-range wireless connection with the wearable device.

In embodiments, each of the at least one wearable device is assigned one of the at least one hub node to have the controller node role for the respective wearable device and be configured for dynamically determining and changing which node is currently having a short-range wireless communication link established with the wearable device.

In embodiments, the at least one hub node having the gatherer node role for a certain wearable device is further configured to determine which of the satellite nodes in the SON is to activate its speaker to play audio data received from the gatherer node over the established audio channel.

In embodiments, the technology disclosed relates to a method in a system including a backend system, a self-organizing network (SON) comprising at least three network nodes including at least one hub node, at least two satellite nodes each comprising a speaker and configured to transmit short-range wireless broadcasts and establish a short-range wireless connection link with a wearable device, and at least one wearable device having short-range wireless communication capabilities and comprising a microphone and at least one of at least one sensor and a user input means, the method comprising:
a. assigning one of the at least one hub node to have the role of a gatherer node for a wearable device;
b. transmitting, by the backend system, downlink data to the SON, wherein said downlink data is at least one of control data and data included in a configuration file;
c. receiving, by the hub node having the gatherer node role for said wearable device, said downlink data originating from the backend system or data corresponding said downlink data originating from the backend system; and
d. triggering, by the hub node currently having the gatherer node role for said wearable device, the initiation of an internal transmission channel or path within the SON which is adapted both for routing sound recorded by the microphone of said wearable device as audio data to said node having the gatherer node role for said wearable device and for routing to the SON incoming audio data of a conference call or call from said gatherer node to one of the satellite nodes so that the audio data can be played out by the speaker of said satellite node, wherein the initiation of the internal transmission channel or path is triggered in response to said received data is indicating at least one of an alert state, a changed physical condition, a changed health state, a changed physical orientation of the person wearing the wearable device and that a call should be established with the wearable device.

In embodiments, the method further comprises:
forwarding, by the hub node having the gatherer node role for said wearable device, audio data recorded by the microphone of said wearable device and transmitted over the internal transmission channel or path within the SON to at least one of an SON-external system managing the conference call or call in which a mobile communication device associated with the wearable device is participating and a mobile communication device associated with the wearable device.

In embodiments, the method further comprises:
transmitting, by the hub node currently having the gatherer node role for said wearable device and in response to said received data originating from the backend system is indicating at least one of an alert state, a changed physical condition, a changed health state, a changed physical orientation of the person wearing the wearable device and that a call should be established with the wearable device, both control data for activating the microphone of said wearable device and control data for activating the speaker of said satellite node.

In embodiments, the method further comprises:
determining, by the hub node currently having the gatherer node role for said wearable device and in response to receiving said data originating from the backend system indicating at least one of an alert state, a changed physical condition, a changed health state, a changed physical orientation of the person wearing the wearable device and that a call should be established with the wearable device, which of the satellite nodes is to activate its speaker to play out audio data received from the conference call or call routed over the internal transmission channel or path, wherein said determining is at least partly based on at least one of signal strength values of short-range wireless signals transmitted by the wearable device and which are measured by at least one of the at least two satellite nodes and then received by the gatherer node via short-range wireless transmissions from the at least one of the at least two satellite nodes, and data related to the wearable device which is collected by at least one other hub node and then shared with the other hub nodes by distribution within the SON.

In embodiments, the method further comprises:
receiving, by the backend system and from the SON, at least one of input data obtained by the user input means of said wearable device and sensor data obtained by at least one sensor of said wearable device; and
transmitting, by the backend system and at least partly based on at least one of input data obtained by the user input means of said wearable device and sensor data obtained by at least one sensor of said wearable device and which is received by the backend system from the SON, said downlink data to the SON.

In embodiments, the method further comprises that the at least one of input data and sensor data obtained by the wearable device is transmitted to the backend system by the one hub node assigned by the SON to have the controller node role for the wearable device, wherein said controller node is responsible for determining and dynamically changing which of the nodes of the SON is currently having the only established short-range wireless connection between the wearable device and the SON.

In embodiments, the method further comprises that the downlink data is received by the one hub node assigned by the SON to have the controller node role for the wearable device and responsible for determining and dynamically changing which only one of the nodes of the SON is currently having an established short-range wireless connection with the wearable device, and wherein the downlink data is shared with the hub node having the gatherer node role for said wearable device.

In embodiments, the method further comprises that each of the at least one wearable device is assigned one of the at least one hub node to have the controller node role for the wearable device and be configured for dynamically determining and changing which node is currently having a short-range wireless communication link established with the wearable device.

In embodiments, the method further comprises that each of the at least one wearable device is assigned one of the at least one hub node to have the controller node role for the wearable device and be configured for dynamically determining and changing which node is currently having the gatherer node role for the wearable device(s) it is responsible for.

In embodiments, the technology disclosed relates to a system including a backend system, a self-organizing network (SON) comprising:
at least three network nodes including at least one hub node,
at least two satellite nodes each comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means,

wherein each of the at least one wearable device is having an established short-range wireless communication link with one of the network nodes and is assigned a hub node to have the role of a gatherer node for the wearable device, and wherein the hub node having the gatherer node role for a wearable device is configured to, in response to receiving downlink data from the backend system indicating that a conference call or call should be established with a wearable device the network node is the gatherer node for, initiate the establishment of an internal transmission channel or path within the SON for routing sound recorded by the microphone of the wearable device as audio data to the gatherer node and for routing to the gatherer node incoming audio data of a conference call or call from the gatherer node to another network node so that the audio data can be played out by the speaker of the other network node.

In embodiments, the backend system is configured to transmit the downlink data in response to receiving uplink data from the SON indicating to the backend system at least one of an alert state, a changed physical condition, a changed health state, a changed physical orientation of the person wearing the wearable device and that a call should be established with the wearable device.

In embodiments, the downlink data received by the SON and the gatherer node is included in at least one of a configuration file which is continuously updated by the backend system and transmitted to the SON, a call initiation request including data for identifying a wearable device which is to participate in a conference call or call, and control data indicating to the hub node having the gatherer node role for a wearable device that the wearable device is to participate in a conference call or call.

In embodiments, the backend system is configured to continuously update data associated with the at least one wearable device based on input data and/or sensor data received by the backend system from the SON, and wherein the downlink data which may trigger the initiation of an internal transmission channel or path within the SON is included in a configuration file continuously updated and transmitted by the backend system to the SON.

In embodiments, the hub node assigned the gatherer node role for a wearable device is configured to initiate the establishment of an internal transmission channel or path within the SON for the purpose of establishing a conference call or call in which the wearable device is to participate in response to the updated and transmitted configuration file is indicating to the gatherer node at least one of an alert state, a changed physical condition, a changed health state, a changed physical orientation of the person wearing the wearable device and that a call should be established with the wearable device. The other network node may then be a satellite node and the hub node having the role of a gatherer node for a wearable device may be configured to initiate the establishment of an internal transmission channel or path to a satellite node which includes at least one short-range wireless communication link between the satellite node and another network node.

In embodiments, the network node having the gatherer node role for a wearable device is configured to use the same internal routing table for initiating the establishment of the internal transmission channel or path for routing audio data obtained by the microphone of the identified wearable device from the other network node to the gatherer node and for routing incoming audio data of a conference call or call to the SON from the gatherer node to the other network node for play out by the speaker of the other network node.

In embodiments, the network node having the gatherer node role for a wearable device may be configured to determine which one of the network nodes is to receive the audio data incoming to the SON through its speaker based on at least one of signal strength values of wireless short-range signals received from the wearable device which are measured by other network nodes and data shared with the gatherer node role by the hub node having the controller node role for the wearable device.

In embodiments, the network node having the gatherer node role for a wearable device may be configured to determine that the one network node currently having an established wireless short-range communication link with the wearable device is the network node for receiving incoming audio data over the internal transmission channel or path and be playing out audio data through its speaker.

In embodiments, the network node currently having an established wireless short-range communication link with the wearable device may be configured to at least one of transmit and forward a request to the wearable device requesting the wearable device to activate its microphone and then transmit sound recorded by the microphone as audio data to the network node currently having an established wireless short-range communication link with the wearable device.

In embodiments, the backend system may be configured to initiate the establishment of a conference call or call in which the wearable device is participating and transmit downlink data, e.g. a call initiation request, in response to receiving uplink data from the SON indicating to the backend system at least one of an alert state, a changed physical condition, a changed health state, a changed physical orientation of the person wearing the wearable device and that a call should be established with the wearable device. The uplink data may then be transmitted to the backend system from the one network node having the controller node role for the wearable device.

In embodiments, the downlink data may be comprised in a call initiation request which comprises at least one of a calling number and SIP credentials (e.g. SIP address and/or SIP username), and wherein the network node having the role of a gatherer node for a wearable device is configured to, in response to receiving the call initiation request from the backend system, activate its audio client to call into a conference and/or call using the at least one of a calling number and SIP credentials.

In embodiments, each of the wearable devices may be configured to receiving a request from the network node currently having an established wireless short-range communication link with the wearable device and, in response to receiving the request, activate its microphone to start recording sound data and transmit recorded sound data as audio data to the network node so that the audio data is transmitted over the internal transmission channel or path within the SON to the network node having the gatherer node role for the wearable device.

In embodiments, the network node having the gatherer node role for a wearable device is configured to determine which of the network nodes is currently having an established wireless short-range communication link with the wearable device.

In embodiments, the network node currently having an established wireless short-range communication link with the wearable device is configured to, in response to receiving a request from the gatherer node, activate its speaker so that incoming audio data from the conference call or call which is transmitted over the internal transmission channel or path is played out by the speaker of the network node currently having an established wireless short-range communication link with the wearable device.

In embodiments, the hub node having the gatherer node role for a wearable device is further configured to forward the audio data recorded by the microphone and received over the internal transmission channel or path within the SON to at least one of an external system managing the conference call or call in which an external mobile communication device associated with the wearable device is participating and a mobile communication device associated with the wearable device which is participating in a call with the wearable device.

In embodiments, the hub node having the gatherer node role for a wearable device is further configured to, in response to receiving downlink data from the backend system which indicates at least one of an alert state, a changed physical condition, a changed health state, a changed physical orientation of the person wearing the wearable device and that a call should be established with the wearable device, transmit control data for instructing the activating the microphone of the wearable device and/or control data for instructing the activation of the speaker of the network node determined to play out the audio data incoming to the gatherer node. The other network node determined to receive and play out the incoming audio data may then be a satellite node the wearable device is currently having an established wireless short-range communication link with. The satellite node may then be further configured to transmit or forward a request to the wearable device instructing the wearable device to activate its microphone and then start transmitting audio data obtained by the microphone over the wireless short-range communication link.

In embodiments, the backend system is configured to transmit the downlink data to the SON and the gatherer node at least partly based on at least one of input data obtained by the user input means of the wearable device and sensor data obtained by at least one sensor of the wearable device and which is received by the backend system from the SON. In embodiments, the at least one of input data and sensor data obtained by the wearable device is transmitted to the backend system by the one hub node assigned by the SON to have the controller node role for the wearable device. The controller node is responsible for determining and dynamically changing which of the nodes of the SON is currently having the only established short-range wireless connection between the wearable device and the SON.

In embodiments, the downlink data received from the backend system is included in at least one of a configuration file which is continuously updated by the backend system and transmitted from the backend system to the SON, and transmitted as control data transmitted downlink from the backend system to the SON.

In embodiments, the downlink data is received by the one hub node having the gatherer node role for the wearable device. The hub node having the gatherer node for a wearable device may be further configured to determine which of the network nodes is to activate its speaker to play out audio data received from the conference call or call routed over the internal transmission channel or path at least partly based on at least one of signal strength values of short-range wireless signals transmitted by the wearable device and which are measured by at least one of the at least two satellite nodes and then received by the gatherer node via short-range wireless transmissions from the at least one of the at least two satellite nodes, and signals strength values and/or data related to the wearable device which is collected by at least one other hub node and then shared with the gatherer node by distribution within the SON.

In embodiments, the hub node having the gatherer node for a wearable device may be further configured to determine which of the nodes is to activate its speaker to play out the incoming audio data from the conference call or call and which is routed over the internal transmission channel or path at least partly based on obtained or received data indicating which of the nodes is the only node in the SON currently having an established short-range wireless connection with the wearable device.

In embodiments, the downlink data is received by the one hub node assigned by the SON to have the controller node role for the wearable device and responsible for determining and dynamically changing which only one of the nodes of the SON is currently having an established short-range wireless connection with the wearable device, and the downlink data is then shared with the hub node having the gatherer node role for the wearable device.

In embodiments, each of the at least one wearable device is assigned one of the at least one hub node to have the controller node role for the wearable device and be configured for dynamically determining and changing which node is currently having a short-range wireless communication link established with the wearable device.

In embodiments, the at least one hub node having the gatherer node role for a certain wearable device is further configured to determine which of the network nodes in the SON is to activate its speaker to play audio data received from the gatherer node over the internal transmission channel or path within the SON.

In embodiments, each of the at least one hub node is configured to continuously receive an updated configuration file from the backend system including status data for each of the at least one wearable device, and wherein the hub node having the gatherer node role for a wearable device is configured to initiate the establishment of an internal transmission channel or path within the SON in response to the updated status data for the wearable device is indicating that the wearable device is to participate in a conference call or call In embodiments, the backend system is configured to, in response to uplink data received from the SON is indicating to the backend system that a conference call or call should be established with one of the at least one wearable device, initiate the establishment of a conference call or call in which the wearable device and a mobile communication device linked to the wearable device in the backend system are to participate.

In embodiments, the backend system is configured to initiate the establishment of a conference call or call by transmitting a request to a third-party call facilitator requesting the third-party call facilitator to start a conference call or call in which the wearable device and a mobile communication device linked to the wearable device in the backend system can call into.

In embodiments, the backend system is configured to initiate the establishment of a conference call or call by transmitting a request to the hub node having the gatherer node role for a wearable device requesting the gatherer node to activate its two-way audio client to call into a conference call or call.

In aspects, the technology disclosed relates to a method in a system including a backend system, a self-organizing network (SON) comprising at least three network nodes including at least one hub node, at least two satellite nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. assigning one of the at least one hub node to have the role of a gatherer node for a wearable device;
b. transmitting, by the backend system, downlink data to the SON;
c. receiving, by the hub node having the gatherer node role for a wearable device, the downlink data from the backend system; and
d. initiating, by the hub node having the gatherer node role for the wearable device and in response to the downlink data from the backend system is indicating to the gatherer node that the wearable device is to participate in a conference call or call, the establishment of an internal transmission channel or path within the SON for routing sound recorded by the microphone of the wearable device as audio data to the node having the gatherer node role for the wearable device and for routing to the gatherer node incoming audio data of a conference call or call from the gatherer node to one of the other network nodes so that the audio data can be played out by the speaker of the other network node.

In embodiments, the method of the technology disclosed comprises that the initiation of the internal transmission channel or path by the gatherer node for the wearable device is triggered in response to the received data is indicating at least one of an alert state, a changed physical condition, a changed health state, a changed physical orientation of the person wearing the wearable device and that a call should be established with the wearable device.

In embodiments, the method of the technology disclosed further comprises:
forwarding, by the hub node having the gatherer node role for the wearable device, audio data recorded by the microphone of the wearable device and transmitted over the internal transmission channel or path within the SON to at least one of an external system for managing the conference call or call in which the wearable device and a mobile communication device associated with the wearable device is participating and a mobile communication device associated with the wearable device.

In embodiments, the method of the technology disclosed further comprising:
transmitting, by the hub node currently having the gatherer node role for said wearable device and in response to the downlink data is indicating that a call should be established with the wearable device, control data for activating the microphone of the wearable device.

In embodiments, the method of the technology disclosed further comprises:
determining, by the hub node having the gatherer node role for the wearable device and in response to the downlink data is indicating that a call should be established with the wearable device, which of the at least three nodes is to activate its speaker to play out audio data received from the conference call or call routed over the internal transmission channel or path.

In embodiments, the determining by the gatherer node which of the at least three nodes is to activate its speaker to play out audio data received from the conference call or call is at least partly based on at least one of signal strength values of short-range wireless signals transmitted by the wearable device and which are measured by at least one of the network nodes and data the wearable device which is collected by at least one other hub node and then shared with the other hub nodes by distribution within the SON.

In embodiments, the method of the technology disclosed further comprises:
receiving, by the backend system and from the SON, at least one of input data obtained by the user input means of the wearable device and sensor data obtained by at least one sensor of the wearable device; and
transmitting, by the backend system and at least partly based on the received input data and/or sensor data obtained by the user input means and/or at least one sensor of the wearable device, the downlink data to the SON.

In embodiments, the at least one of input data and sensor data obtained by the wearable device is transmitted to the backend system by the one hub node assigned by the SON to have the controller node role for the wearable device, wherein said controller node is responsible for determining and dynamically changing which of the nodes of the SON is currently having an established short-range wireless connection with the wearable device.

In embodiments, the method of the technology disclosed the downlink data is received by the one hub node assigned by the SON to have the gatherer node role for the wearable device and thereby responsible for initiating the internal transmission channel or path and for determining which of the at least three nodes is to activate its speaker to play out audio data received from the conference call or call.

In embodiments, the method of the technology disclosed comprises that each of the at least one wearable device is assigned one of the hub nodes to have a controller node role for the wearable device, and wherein the hub node having the controller node role for a wearable device is responsible for dynamically assigning one of the network nodes to have the gatherer node role for the wearable device it is responsible for.

In embodiments, the downlink data is received by the one hub node assigned by the SON to have the controller node role for the wearable device and responsible for determining and dynamically changing which only one of the nodes of the SON is currently having an established short-range wireless connection with the wearable device, and wherein the controller node is configured to share the received downlink data with the hub node having the gatherer node role for the wearable device.

In embodiments, the method includes that each of the at least one wearable device is assigned one of the at least one hub node to have the controller node role for the wearable device and be configured for dynamically determining and changing which node is currently having a short-range wireless communication link established with the wearable device.

In embodiments, each of the at least one wearable device is assigned one of the at least one hub node to have the controller node role for the wearable device and be configured for dynamically determining and changing which node is currently having the gatherer node role for the wearable device(s) it is responsible for.

In embodiments, the method of the technology disclosed further comprises:
initiating, by the backend system and in response to uplink data received from the SON is indicating to the backend system that a conference call or call should be established with one of the at least one wearable device, a conference call or call in which the wearable device and a mobile communication device linked to the wearable device in the backend system are to participate.

In embodiments, the method of the technology disclosed further comprises initiating, by the backend system, the establishment of a conference call or call by transmitting a request to a third-party call facilitator requesting the third-party call facilitator to start a conference call or call in which the wearable device and a mobile communication device linked to the wearable device in the backend system can call into.

In embodiments, the method of the technology disclosed further comprises initiating, by the backend system, the establishment of a conference call or call by transmitting a request to the hub node having the gatherer node role for a wearable device requesting the gatherer node to activate its two-way audio client to call into the conference call or call.

In embodiments, the technology disclosed provides a fast response yet robust solution for acting on an identified event by parallel redundant data transmission of event data representing or reflecting the event via both an established short-range wireless connection with one of the nodes and via short-range wireless broadcast messages from the personal device to the nodes which are within short-range wireless communication range.

Bluetooth is a short-range wireless technology standard that allows data to be exchanged between fixed and mobile devices. The technology uses short wavelength radio waves from 2.4 to 2.485 GHz. Unlike other forms of connectivity such as wi-fi or 4G, Bluetooth carries connections between devices and other devices as opposed to carrying data to and from the internet.

BLE stands for Bluetooth Low Energy and is a form of wireless communication designed especially for short-range communication. BLE is very similar to Wi-Fi in the sense that it allows devices to communicate with each other. However, BLE is meant for situations where battery life is preferred over high data transfer speeds. Wi-Fi uses multiple parts of the IEEE 802 protocol family and is designed to interwork seamlessly with its wired sibling Ethernet. Compatible devices can network through wireless access points to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 GHz (120 mm) UHF and 5 GHz (60 mm) SHF ISM radio bands; these bands are subdivided into multiple channels. Channels can be shared between networks but only one transmitter can locally transmit on a channel at any moment in time.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location, e.g. current room, for a personal device without any use of high energy-consuming GPS in the personal devices or iBeacon technology, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

The controller node, or a local or remote processing arrangement receiving the measured signals strengths from the controller node (and optionally also sensor data obtained by a sensor of the personal device), may then be configured to determine the current position or location for the personal device by comparing the signal strengths of the broadcast signals measured by the other nodes (and the controller node) and then determine the position or location based on the comparison. In embodiments, the position or location of the node measuring the highest signal strength for the broadcast signal is determined to be the position or location for the personal device, or the position or location for the personal device may be determined or calculated using signals strengths measured by a plurality of nodes, e.g. by using triangulation, or the position or location for the personal device may be determined or calculated using at least one signals strength of a broadcast signal and sensor data obtained by a sensor of the personal device, e.g. a motion sensor or a pressure sensor.

In certain embodiments, the current position or location for the personal device may at least partly be determined by a trained machine learning algorithm used by the controller node or a backend system communicatively coupled to the controller node, since machine learning is an efficient way of evaluating obtained sensor data and event data, e.g. in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically and continuously changing the connection node for a personal device, by measuring, by each of the network nodes which are within short-range communication range with the personal device, the signal strength of short-range wireless broadcast messages, e.g. Bluetooth broadcast messages, received from the personal device.

In certain embodiments, the setting of an alarm or the change of health state and/or alert state for the personal device may at least partly be determined by a trained machine learning algorithm used by the controller node or a backend system communicatively coupled to the controller node, since machine learning is an efficient way of evaluating sensor data in situations where large amounts of data is collected. The trained machine learning algorithm may then use at least the signal strength values received from the nodes (including the current controller node and the current connection node for the wearable device if any of these nodes are within short-range wireless communication range with the wearable device to receive the broadcasts) and/or movement sensor data detected by the personal device to determine that the connection node for the wearable device should be changed.

FIG. 1 schematically shows an example of a facility 700 where a system 100 comprising a number of network nodes (301, 302, 303, 304, 305, 306) has been installed, in accordance with one or more embodiments described herein. The illustrated system 100 comprises a plurality of wearable devices (201, 202, 203, 204, 205) carried by individuals and having short-range wireless communication capabilities. Such facility 700 may e.g. be an apartment where a monitored individual is residing. Such an apartment 700 may e.g. comprise a kitchen 710, a bathroom 720, a bedroom 730, a sitting room 740, and a hallway 750. The system 100 may in this case be used by relatives, home care companies or the like.

The facility 700 may also be other types of quarters where a monitored individual may be residing, such as e.g. a nursing home, where there may be a private space for the monitored individual, including e.g. a bedroom 730, as well as public spaces that are shared by a plurality of monitored individuals.

In this example embodiment of the technology disclosed, the network nodes with reference signs 301, 302 and 303 in FIG. 1 are hub nodes each configured to share data, including data associated with the wearable devices, with each other via established communication links between the nodes. In this example embodiment, each of the hub nodes (301, 302, 303) comprises a speaker for playing out audio data received from a conference call or call. The network nodes with reference signs 304, 305, 306 and 307 are satellite nodes each comprising a speaker for playing out audio data received from a conference call or call and further configured to transmit short-range wireless broadcasts and to establish a short-range wireless communication link with a wearable device.

The wearable devices (201, 202, 203, 204, 205) are each connected to the network via an established short-range wireless communication link with one of the network nodes (301, 302, 303, 304, 305, 306, 307). The wearable devices (201, 202, 203, 204, 205) in FIG. 1 each have short-range wireless communication capabilities and comprises a microphone, one or two sensor(s), and a user input means. The wearable devices (201, 202, 203, 204, 205) are each configured to transmit short-range wireless broadcasts signals that may be received by any of the network nodes (301, 302, 303, 304, 305, 306, 307) that currently is within short-range wireless communication range with the wearable device transmitting the broadcasts.

The wearable device with reference sign 202 in FIG. 1 is currently connected to the network via a short-range wireless communication link with the satellite node with reference sign 306 and the wearable device with reference sign 201 in FIG. 1 is currently connected to the network via a short-range wireless communication link with the hub node with reference sign 301. The hub nodes (301, 302, 303) are each configured to both continuously share their data with other hub nodes and to establish short-range wireless communication links with each of the wearable devices.

The hub node with reference sign 301 is having the controller node role for the wearable device with reference sign 202, thereby being responsible for collecting sensor data and input data obtained by the wearable device 202, further being responsible for assigning which of the network nodes is having the gatherer node role for the wearable device 202 and for dynamically determining and changing which of the network nodes is currently having the only one established short-range wireless communication link with the wearable device 202.

In the example embodiment illustrated in FIG. 1, only the hub nodes (301, 302, 303) can have the roles of controller node and gatherer node for a wearable device (201, 202, 203, 204, 205). The hub node 301 in FIG. 1 can determine which of the hub nodes network nodes (301, 302, 303), including itself, is the gatherer node for the wearable device 202 and to determine and change which of the network nodes (301, 302, 303, 304, 305, 306), including itself, is currently having an established short-range wireless communication link with the wearable device 202. In this example, hub node 302 has been assigned by the controller node 301 to be the gatherer node for the wearable device 202 and satellite node 306 is assigned by the controller node 301 to be connection node for the wearable device having the only one established short-range wireless communication link with the wearable device 202.

The hub node 302 having the gatherer node role for the wearable device 202 initiated, in response to receiving downlink data from a backend system (not shown) indicating that a conference call or call should be established with the wearable device 202, the establishment of an internal transmission channel/path within the network for routing sound recorded by the microphone of the wearable device 202 as audio data to the gatherer node 302 and for routing to the gatherer node incoming audio data of a conference call from the gatherer node to the satellite node 306 so that the audio data from the conference call can be played out through the speaker of the satellite node 306. According to this embodiment, the satellite node 306 was determined by the gatherer node 302 to be the network node for playing out the audio data from the conference call through its speaker because it was the network node currently having an established short-range wireless communication link with the wearable device 202 and still measuring relatively high signal strength values of signals transmitted by the wearable device 202, i.e. the satellite node 306 was the node dynamically assigned by the controller node 301 to be the connection node for the wearable device 202. The established internal transmission channel/path in FIG. 1 goes from the gatherer node 302, via satellite nodes 305 and 304 to the satellite node 306 which is the current connection node for the wearable device 202 having a short-range wireless communication link with the wearable device 202.

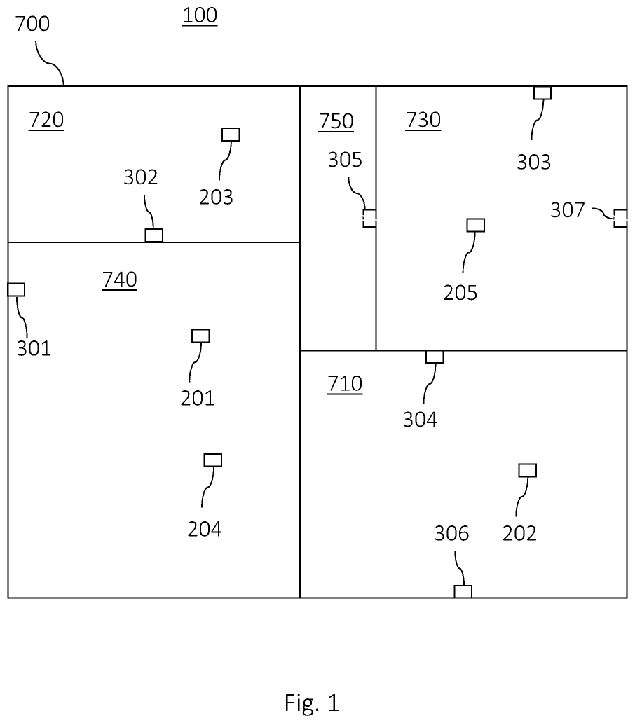

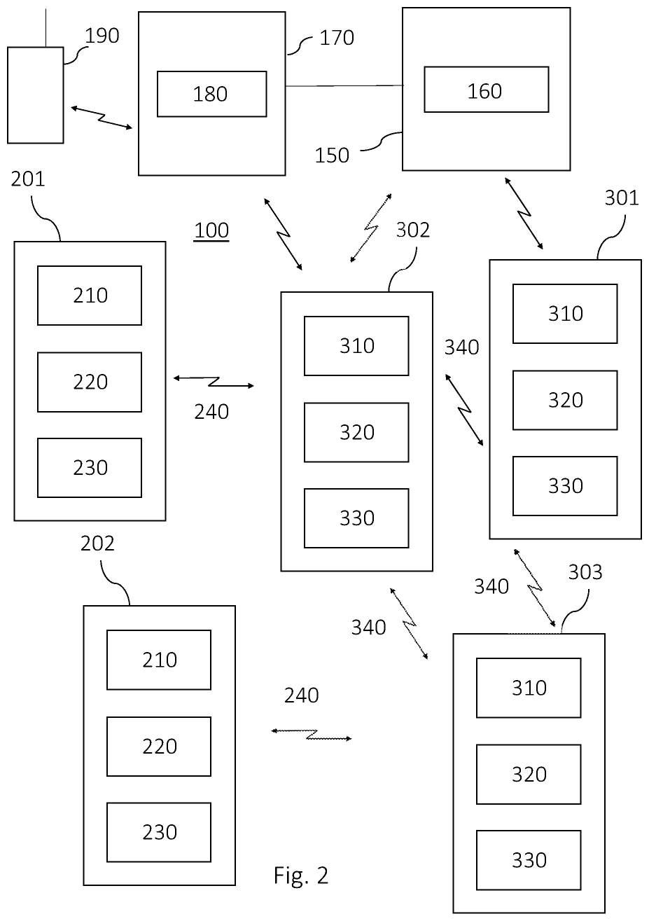

FIG. 2 is a schematic illustration of a system 100 for monitoring an individual within predetermined facilities 700, in accordance with one or more embodiments described herein. The illustrated system 100 comprises a plurality of wearable devices (201, 202), a plurality of network nodes (301, 302, 303), a processing arrangement 150, e.g. a remote processing arrangement such as a backend system, and an external system 170 for managing and an established call in which a wearable device 202 and a mobile communication device 190 associated with the wearable device 202 is participating. The wearable devices (201, 202) in FIG. 2 are wristbands worn by two separate individuals, e.g. a necklace or a clip worn by two patients in a nursing home 700.

The wearable devices (201, 202) illustrated in FIG. 2 each comprises a microphone 210, user input means 220, and at least one sensor 230. The at least one sensor 230 may, for example, include a motion sensor, a temperature sensor, a health sensor such as a body temperature sensor, a pressure sensor and/or any other type of sensor and the user input means 220 may comprise a button or voice input means that the individual can press or activate to set off an alarm and/or transmit event data indicating the occurrence of a certain event. The illustrated network nodes (301, 302, 303) comprise a speaker 310, a storage means 320, and a node communication interface 330. The illustrated processing arrangement 150 comprises a processing device 160, which may e.g. be comprised in a web server.

In FIG. 2, the network node with reference sign 302 having the gatherer node role for the wearable device with reference sign 202 initiates, in response to receiving downlink data from a backend system 150 indicating that a conference call should be established with the wearable device 202, the establishment of an internal transmission channel or path within the network for routing sound recorded by the microphone 210 of the wearable device 202 as audio data to the gatherer node 302 and for routing to the gatherer node 302 incoming audio data of the conference call from the gatherer node to another network node 303 so that the audio data can be played out by the speaker 310 of the network node 303.

The network node 302 in FIG. 2 having the gatherer node role for the wearable device 202 further forwards the audio data recorded by the microphone 210 of the wearable device 202 and received over the internal transmission channel or path within the network to an external system 170 including processing means 180 for managing the conference call in which an external mobile communication device 190 associated with the wearable device 202 and the wearable device 202 are both participating in.

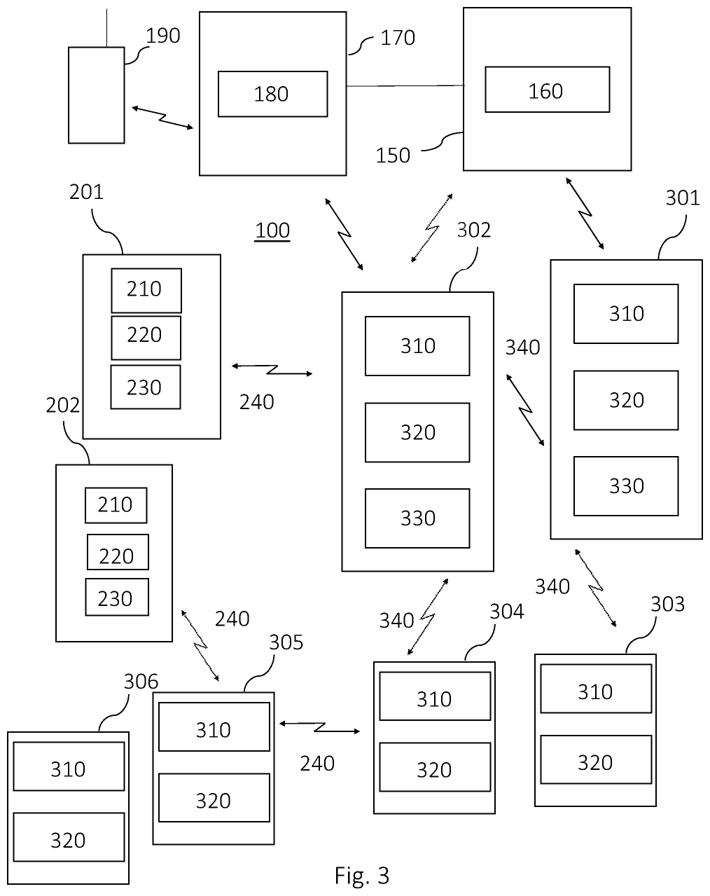

FIG. 3 is a schematic illustration of a system 100 for monitoring an individual within predetermined facilities 700, in accordance with one or more embodiments described herein. The example system 100 illustrated in FIG. 3 comprises two hub nodes comprising a speaker 310 and configured to have a controller node role or gatherer node role for one of the wearable devices 201 and 202 and four satellite nodes comprising a speaker 310 and configured to both transmit short-range wireless broadcast signals and listen for short-range wireless broadcast signals transmitted by other devices and nodes.

The wearable devices (201, 202) illustrated in FIG. 3 each comprises a microphone 210, user input means 220, and at least one sensor 230. The at least one sensor 230 may, for example, include a motion sensor, a temperature sensor, a health sensor such as a body temperature sensor, a pressure sensor and/or any other type of sensor and the user input means 220 may comprise a button or voice input means that the individual can press or activate to set off an alarm and/or transmit event data indicating the occurrence of a certain event. The illustrated network nodes (301, 302, 303, 304, 305, 306) each comprises a speaker 310 and a node communication interface 330. The example hub nodes illustrated in FIG. 3 each comprises a storage means 320 for storing, for example, sensor data obtained by a wearable device the respective hub node is responsible for. The illustrated processing arrangement 150 comprises a processing device 160, which may e.g. be comprised in a web server.

In FIG. 3, the hub node 302 having the gatherer node role for the wearable device 202 initiates, in response to receiving downlink data from a backend system 150 indicating that a call should be established with the wearable device 202, the establishment of an internal transmission channel or path within the network for routing sound recorded by the microphone 210 of the wearable device 202 as audio data to the gatherer node 302 and for routing to the gatherer node 302 incoming audio data of the call from the gatherer node via a first satellite node 304 to a satellite node 305 so that the audio data can be played out by the speaker of the satellite node 305.

The hub node 302 in FIG. 3 having the gatherer node role for the wearable device 202 further forwards the audio data recorded by the microphone and received over the internal transmission channel or path within the network an external third-party system 170 including processing means 180 for managing the call in which an external mobile communication device 190 associated with the wearable device 202 and the wearable device 202 are participating in.

Information regarding the layout of the facilities 700, such as the plan of the different rooms and the location of the node modules 300, is preferably stored in the storage means 320. The storage means 320 does not have to be comprised in the network node—it can be comprised in another part of the system such as a locally-installed processing arrangement or a remote processing arrangement such as a backend system 150 or be a separate device or module.

According to embodiments of the technology disclosed, the network node with reference sign 301 in FIG. 3 is a hub node having the controller node role for the wearable device 202 and is therefore arranged to: receive status data, position data or signal strength values from the other hub nodes (302, 303) of the network, or sensor data, input data or event data originating from the at least one sensor 230 and/or user input means 220 of the wearable devices 202; determine whether the received data indicates a need for certain action associated with the wearable device 202, e.g. changing the health state for the individual wearing the personal device and/or alert state among a plurality of predetermined alert states. As an example, the alert states may comprise at least a FALL alert state, to be used if a probable fall has been detected for the monitored individual, and an OUT OF BED alert state, to be used if it has been detected that the monitored individual is probably getting out of bed. The hub nodes (301, 302, 303) in FIG. 3 receive sensor data and input data from the wearable device 202 which is received in broadcasts from the wearable device 202 by any satellite node (303, 304, 305, 306) within short-range communication range with the wearable device 202 and forwarded by the respective satellite node (303, 304, 305, 306) via short-range wireless transmissions to the hub nodes (301, 302, 303).

If weighing factors are used, these weighing factors may be determined by a machine learning system, since this is an efficient way of evaluating complex signals in situations where large amounts of data is collected.

One way of making such a determination is to determine a probability that an alert should be set based on the received event data and/or sensor data, and a probability that the determined location fulfils the predetermined location condition, and then combine these probabilities using weighing factors, which may e.g. be determined by a machine learning system.

The same applies to other combinations, such as e.g. the determination of the position and/or location for the individual wearing the personal device by involving also signals from other sensors of the personal device such as a movement sensor 230, and/or the setting of alerts based also on activity states. These determinations may also be done by determining probabilities and using weighing factors to combine them, which may e.g. be determined by a machine learning system.

In embodiments, the determining of a position and/or location for the individual wearing the personal device by the controller node (301) may e.g. be based on the received signal strength indication (RSSI) of short-range wireless broadcast signals transmitted by the wearable device 202 and which are received by the network nodes within short-range communication range with the personal device. The network nodes within short-range communication range then measure the RSSI of short-range wireless broadcast signals and forwards the RSSI to the controller node 301 for the wearable device 202. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by the wearable device 202 may be used by the controller node 301 assigned to the wearable device 202 for determining the current position and/or location of the wearable device 202 in connection with transmitting position or location data together with alert state data or when setting an alert or alarm for the individual wearing the wearable device 202. Thus, the decisions by the controller node 301 for the particular wearable device 202 which of the network nodes is playing out audio data from the conference call or call received via the internal transmission channel or path through its speaker, to set an alarm for the individual carrying the wearable device 202 or to transmit alert state data or alarm data may be based on broadcast signal strength values measured and received from other nodes within short-range communication range with the wearable device (including the current controller node and the current connection node for the personal device if any of these nodes are within short-range wireless communication range with the wearable device 202 to receive the broadcasts).

The wearable devices (201, 202, 203, 204, 205) illustrated in Figures may also comprise other sensors 230, such as e.g. a pressure sensor, temperature sensor or medical sensor. It is known to use a pressure sensor in a personal device to detect a fall based on a change in pressure. However, a pressure sensor may also be involved in the determination of the location of the individual, e.g. when location data is transmitted together with health state data and/or alert state data to a local or remote processing arrangement 150, e.g. a backend system, configured to set an alert or an alarm. If the facilities 700 comprise several floors, and the network nodes are arranged in the ceilings or high up on the walls, the RSSI may give erroneous results due to the signals travelling between the floors. In this situation, a pressure sensor may be used to determine the floor on which the individual is located.

In order to improve the determination of the need for e.g. changing health state for the individual and/or setting an alert even further, the determination of the location may be improved by involving also signals from a movement sensor 230. The movement sensor 230 may e.g. be used as a pedometer, so that the signals from the movement sensor 230 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 230, it is likely that the determined location is incorrect. The at least one processing device comprised in at least one of a network node or in the backend system 160 may thus be arranged to determine the location of the wearable device 200 based also on an indication of the movement of the individual in relation to a previously determined location, based on sensor data from the at least one motion sensor 230.

The processing of the sensor data from the at least one sensor 230 may be done by a trained machine learning algorithm used by the controller node or a local or remote processing arrangement communicatively connected to the personal device. When many individuals are monitored using many personal devices 200, large amounts of sensor data is collected from the sensors 230 in these personal devices 200. Very efficient evaluation of this sensor data may be done using machine learning, in order to determine a suitable machine learning algorithm.

In certain embodiments, the determining of which of the network nodes is playing out audio data from the conference call or call through its speaker is at least partly based at least on the received signal strength values together with the information retrieved from a storage means 320 regarding the layout of the facilities 700 within which the monitoring of the individual takes place.

In the embodiments, the decision to establish a call and the decision which of the network nodes is to play out audio data from the call through its speaker may be based at least partly on received sensor data, e.g. obtained by a temperature sensor, pressure sensor or motion sensor of the wearable device. In various embodiments, the actual decision to establish a conference call or call may then be taken by a backend system or the network node having the controller node role for the wearable device. The determining which of the network nodes is to play out incoming audio data from the conference call or call transmitted over the internal channel or path through its speaker may be taken by the network node having a gatherer node role for the wearable device or the controller node for the wearable device which informs the gatherer node about its decision by transmitting certain data to the gatherer node.

The combination of motion information with location information improves the determination of changing connection node for the wearable device.

The determining of whether the received sensor data indicates a specific location among a plurality of predetermined location may e.g. be done by a trained machine learning algorithm of the controller node or a locally-installed processing arrangement or a remote processing arrangement such as a backend system communicatively connected to the controller node, since machine learning is an efficient way of evaluating complex signals in situations where large amounts of data is collected and moving patterns of the individuals wearing the personal devices are repetitive and/or predictable.

In embodiments, the distance between the personal device 200 and the at least one network node 300 may e.g. be determined based on the received or obtained signal strength indication (RSSI) of the short-range wireless signal transmitted by the personal device 200 and received by the plurality of network nodes, since the received signal strength will be lower if the distance increases. If the RRSI for more than one network node is determined, the location may be more exactly determined based on e.g. triangulation. Machine learning may also be used for this determination.

In embodiments, the determining, by the controller node and/or gatherer node for a wearable device to participate in a conference call or call, of which of the network nodes will be playing out audio data from the conference call or call may at least partly be based also on the current position, location and/or an indication of the movement of the individual in relation to a previously determined location, based on sensor data from the at least one sensor of the wearable device. If the individual is determined to be in a certain location, and the movement required for moving to this location from a previously determined location is more than the movement that has been detected by the at least one movement sensor, it is likely that the determined location is incorrect.

In embodiments, the at least one sensor 230 is an accelerometer. However, other types of motion sensors may also be used.

In embodiments, the personal device 200 is a wristworn device, such as e.g. a wristband. The personal device 200 may however be any type of device that may be worn by an individual, such as e.g. a necklace or a clip.

In embodiments, communication between the wearable device 200 and the at least one node 300 takes place using a personal device communication interface and a node communication interface. In embodiments, the technology disclosed may include continuously determining and updating an activity state for the monitored individual by continuously receiving sensor data from the personal device, e.g. health sensor data obtained by a medical sensor of the personal device.

The activity state may e.g. categorize the current activity of the individual. Activity states may e.g. be LYING IN BED, LYING ON COUCH, LYING ON FLOOR, SITTING, STANDING, WALKING, RUNNING, RIDING A WHEELCHAIR, WALKING UP/DOWN STAIRS, IN THE BATHROOM. There may also be a specific activity state for when the individual is not wearing the personal device, since no other activity state can then be set. There may be a specific sensor, such as e.g. a contact sensor, a temperature sensor or a heart rate sensor, for determining whether the individual is wearing the personal device. Such a sensor may of course also be used for other purposes.

In embodiments, the technology disclosed may include continuously determining and updating a health state for the monitored individual by continuously receiving sensor data from the personal device worn by the individual, e.g. health sensor data obtained by at least one medical sensor of the personal device.

The determining of the activity state and/or the health state of the individual wearing the personal device may e.g. be done by a trained machine learning algorithm, since machine learning is an efficient way of evaluating complex signals in situations where large amounts of data is collected.

In embodiments, the wearable device 200 may also comprise sensors, such as e.g. a motion sensor or pressure sensor. It is known to use a pressure sensor in a personal device to detect a fall based on a change in pressure. However, a pressure sensor may also be involved in the determination, by the controller node, to change gatherer node for the personal device 200 carried by the individual. If the facilities 700 comprise several floors, and the network nodes 300 are arranged in the ceilings or high up on the walls, the RSSI may give erroneous results due to the signals travelling between the floors. In this situation, data obtained by a pressure sensor of the personal device received by the controller node may be used by the controller node to determine the floor on which the individual is located which, in turn, is an input factor to the step of determining, by the controller node for the personal device 200, whether to change connection node for the personal device 200.

In embodiments and in order to improve the determination of whether to change connection node for a personal device 200, the determination of the location and/or whether to change connection node may be improved by involving also signals from a sensor 230 of the personal device 200. The sensor 230 may e.g. be a movement sensor used as a pedometer, so that the signals from the movement sensor 230 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 230, it is likely that the determined location is incorrect. The at least one processing device of a network node (301, 302, 303, 304, 305, 306) may thus be arranged to determine the location of the personal device 200 influencing the decision by the controller node, or processing arrangement, whether to change alert state and/or health state for the personal device 200 based also on an indication of the movement of the individual in relation to a previously determined location, based on sensor data from the at least one sensor 230.

The processing of the sensor data from the at least one sensor 230 may be done by a trained machine learning algorithm. When many individuals are monitored using many personal devices 200, large amounts of sensor data is collected from the sensors 230 in these personal devices 200. Very efficient evaluation of this sensor data may be done using machine learning, in order to determine a suitable machine learning algorithm.

The updating of the activity state and/or health state for the monitored individual may be incremental, so that only differences in relation to the previously determined activity state and/or health state need to be determined.

The determination of the location for the personal device 200 and/or the by the controller node to change connection node for the personal device 200 may e.g. be done by a trained machine learning algorithm. Machine learning is an efficient way of evaluating complex signals in situations where large amounts of data is collected.

When the determining of whether to change alert state and/or health state is based on received sensor data indicates a certain location and whether the determined location fulfils a predetermined location condition, the predetermined location condition may be an absolute condition, such that if the determined location does not fulfil the predetermined location condition, the controller node will, or will not, determine to change alert state and/or health state for the personal device 200. However, there may instead be weighing factors, so that if the received sensor data strongly indicates a specific location, and/or the determined location does not strongly indicate a specific location (that does not fulfil the predetermined location condition), the controller node will, or will not, determine to change alert state and/or health state for the personal device 200.

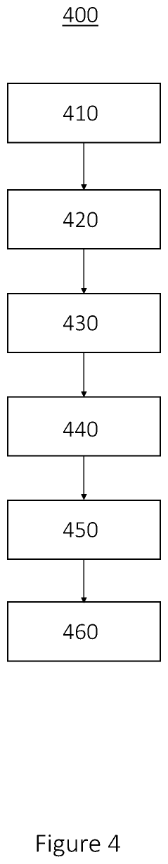

FIG. 4 is an example flow diagram of a method for monitoring an individual within predetermined facilities in order to determine whether there is a need for establishing, by the gatherer node, an internal transmission channel or path for the transmission of audio data of a conference call or call involving a wearable device carried by the individual in response to status data, sensor data and/or input data received by the controller node for the wearable device, e.g. in response to a changed health state or changed position and/or body position for the individual and/or setting an alert, in accordance with one or more embodiments described herein.

FIG. 4 schematically illustrates embodiments of the technology disclosed including a method (400) for monitoring an individual wearing a wearable device connected to a network for monitoring the individual within predetermined facilities (700), the method (400) comprising:
a. assigning (410) one of the at least one network node to have the role of a gatherer node for a wearable device;
b. transmitting (420), by the backend system, downlink data to the network;
c. receiving (430), by the network node having the gatherer node role for a wearable device, the downlink data from the backend system;
d. initiating (440), by the network node having the gatherer node role for the wearable device and in response to the downlink data from the backend system is indicating to the gatherer node that the wearable device is to participate in a conference call or call, the establishment of an internal transmission channel or path within the network for routing sound recorded by the microphone of the wearable device as audio data to the node having the gatherer node role for the wearable device and for routing to the gatherer node incoming audio data of a conference call or call from the gatherer node to one of the other network nodes so that the audio data can be played out by the speaker of the other network node,
e. receiving (450), by the network node having the gatherer node for the wearable device, audio data recorded by the microphone of the wearable device, wherein the audio data is transmitted from the wearable device to the gatherer node via the established internal transmission channel or path, and
f. forwarding (460), from the gatherer node to an external system for managing the conference call or call, the audio data received over the established internal transmission channel or path.

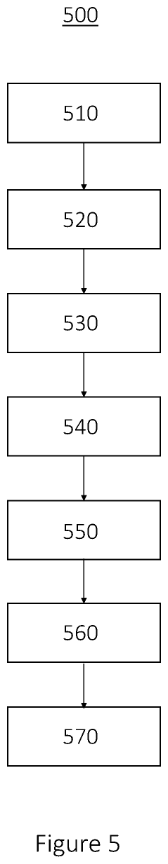

FIG. 5 schematically illustrates embodiments of the technology disclosed including a method (500) for monitoring an individual within predetermined facilities (700) and determining which of the nodes of a network of nodes is to play out incoming audio data of a conference call or call received from an external third-party system, the method (500) comprising:
a. transmitting (510), by a wearable device carried by the individual, short-range wireless broadcast signals;
b. receiving (520), by network nodes within short-range wireless communication range with the wearable device, at least one of the short-range wireless broadcast signals;
c. measuring (530), by each of the network nodes within short-range wireless communication range, signal strengths of the received at least one short-range wireless broadcast signal;
d. transmitting (540), continuously from each of the other network nodes and via the node network connecting the at least three nodes of the node network, data to the controller node for the wearable device, wherein the data includes data indicating the measured signal strengths values and at least one of input data obtained by a user input means of the wearable device and/or sensor data obtained by a sensor of the wearable device;
e. transmitting (550), by the controller to the gatherer node assigned to the wearable device by the controller node, data including at least one of sensor data obtained by a sensor of the wearable device, measured signal strength values, data indicating which of the network nodes is currently the connection node for the wearable device, and data indicating to the gatherer node which of the network nodes is playing out through its speaker audio data from a conference call or call in which the wearable device is to participate; and
f. determining (560), by the gatherer node at least partly based on said data received from the controller node, which of the network nodes including itself is to receive the audio data from the conference call or call, via an established internal transmission channel or path, and
g. playing out (570), by the network node determined to be receiving the audio data from the conference call or call via an established internal transmission channel or path, the audio data through its speaker.

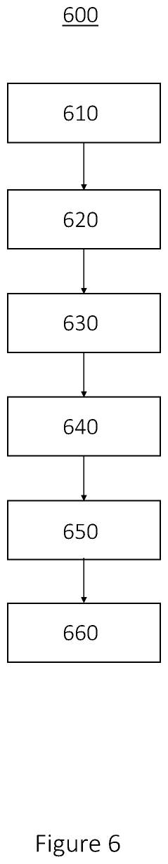

FIG. 6 schematically illustrates embodiments of the technology disclosed including a method (600) for monitoring an individual within predetermined facilities (700), the method (600) comprising:
a. transmitting (610), by a wearable device carried by the individual, short-range wireless broadcast signals;
b. receiving (620), by network nodes within short-range wireless communication range with the wearable device, at least one of the short-range wireless broadcast signals;
c. measuring (630), by each of the network nodes within short-range wireless communication range, signal strengths of the received at least one short-range wireless broadcast signal;
d. transmitting (640), from each of the plurality of nodes and via the node network connecting the at least three nodes of the node network, data to the controller node, wherein the data indicates the measured signal strengths values;
e. receiving (650), by the controller node assigned to the wearable device and via the node network, the transmitted data indicating the measured signal strengths values; and
f. determining (660), by the controller node, to transmit data to a processing arrangement and/or set an alert state or health state for the wearable device and/or the individual wearing the personal device at least partly based on the received data indicating the measured broadcast signal strengths values.

In aspects, the technology disclosed relates to a method and system for monitoring an individual within predetermined facilities (700):
receiving, by the sole connection node assigned to the personal device, a message comprising sensor data obtained by at least one sensor (230) arranged in the personal device (200);
forwarding, by the connection node and to the controller node assigned to the personal device, a message comprising the sensor data or data reflecting the sensor data;
determining, by the controller node whether the received sensor data indicates certain event data, a specific health state and/or a specific alert state among a plurality of predetermined alert states, where the alert states may comprising at least a FALL alert state, to be used if a probable fall has been detected for the monitored individual, and an OUT OF BED alert state, to be used if it has been detected that the monitored individual is probably getting out of bed;
receiving, by a plurality of nodes within short-range wireless communication range with the personal device, short-range wireless broadcast signals from the personal device;
measuring, by each of the plurality of nodes, signal strengths values of the received short-range wireless broadcast signals;
forwarding, by each of the plurality of nodes and to the controller node assigned to the personal device, a message containing the signal strengths values of the received short-range wireless broadcast signals; determining, by the controller node, the location of the personal device (200) within the facilities (700) based at least on the received signal strengths values of the short-range wireless broadcast signals together with information retrieved from a storage means (320) regarding the layout of the facilities (700) within which the monitoring of the individual takes place; and
setting an alert and/or change a health state based at least on whether the received sensor data indicates a specific event, health state and/or an alert state and whether the determined location fulfils a predetermined location condition, indicating whether the individual is located in a certain room, such as e.g. the bedroom (630).

According to embodiments of the various methods mentioned above, the determining of the location of the personal device (200) is based also on an indication of the movement of the individual in relation to a previously determined location, based on sensor data from the at least one sensor (230), e.g. a motion sensor and/or a pressure sensor.

According to embodiments of the technology disclosed and mentioned above, the method further comprising continuously determining and updating, by the controller node for the wearable device carried by an individual, an activity state and/or a heath state for the monitored individual.

According to embodiments of the technology disclosed and mentioned above, the method further comprising continuously determining and updating, by the backend system and based on data received from the controller node for the wearable device carried by an individual, an activity state and/or a heath state for the monitored individual.

According to embodiments of the technology disclosed and mentioned above, the method further the change of health state and/or setting of the alert is based also on at least one previously determined health state or activity state. In the example of setting an alert state, the setting of an OUT OF BED alert state may also depend on whether the previous activity state was a LYING IN BED alert state, or so that the setting of a FALL alert state also depends on that the previous activity state was not any type of LYING activity state.

According to embodiments of the technology disclosed and mentioned above, the changed health state and/or setting of the alert is based also on the time period for which the individual has had a specific activity state, such as e.g. for how long the individual has been in the activity state STANDING.

According to embodiments of the technology disclosed and mentioned above, determining of the changed health state and/or activity state is done by a trained machine learning algorithm.

According to embodiments of the technology disclosed and mentioned above, the method further comprises sending an alarm signal if a predetermined alert or health state is set.

In embodiments, the communication between the personal device (200) and the at least one network node (300) takes place using a personal device communication interface and a network node communication interface (330).

In aspects, the technology disclosed relates to a system (100) for monitoring an individual within predetermined facilities (700), the system (100) comprising:
a wearable device (200) to be worn by the monitored individual, the personal device (200) comprising at least one sensor (230) or input means (220);
at least one network node (300), with which the personal device (200) is arranged to communicate via an established short-range wireless connection;
a storage means (320); and
at least one processing device (160), arranged to:
   receive sensor data, event data and/or input data from the at least one sensor (230) and/or input means (220) of the wearable device (200);
   determine whether the received sensor data, event data and/or input data indicates a specific health state and/or an alert state among a plurality of health states and/or predetermined alert states; and;
   set a health state and/or an alert based at least on whether the received sensor data, event data and/or input data indicates a health state and/or an alert state.

In embodiments, the system (100) and controller node for the wearable device (200) are configured to set a health state and/or an alert based at least on whether a determined location fulfils a predetermined location condition, indicating whether the individual is located in a certain room, such as e.g. the bedroom (630).

In embodiments, the system (100) and controller node for the wearable device (200) are configured to determine the distance between the personal device (200) and the at least one network node (300), e.g. based on signal strength values measured by at least one of the network nodes.

In embodiments, the system (100) and controller node for the personal device (200) are configured to determine the location of the personal device (200) within the facilities (700) based at least on the determined distance together with information retrieved from the storage means (320) regarding the layout of the facilities (700) within which the monitoring of the individual takes place.

In embodiments, the system (100) and the at least one processing device (160) are arranged to continuously determine and update a health state and/or an activity state for the monitored individual based on received sensor data is indicating a specific event, health state and/or an alert state.

In embodiments, the personal device (200) comprises at least one motion sensor (230). In certain embodiment the at least one motion sensor (230) is an accelerometer.

In embodiments, the personal device (200) comprises at least one medical sensor (230), or health sensor, where the medical sensor (230) is adapted to obtain health sensor data to be used for determining the current health status or health state of the individual wearing the personal device.

In embodiments, the personal device (200) is a wristworn device, such as e.g. a wristband, clip or necklace.

An accelerometer, is a device that measures proper acceleration. Proper acceleration, being the acceleration (or rate of change of velocity) of a body in its own instantaneous rest frame, is not the same as coordinate acceleration, being the acceleration in a fixed coordinate system.

The input from the movement sensor based on sensor data indicating an acceleration beyond a certain threshold value and/or a certain changed movement pattern for the personal device may trigger the short-range wireless transmission of event data from the personal device.

1: A system for monitoring individuals including a backend system, a self-organizing network (SON) comprising:
at least three network nodes including at least one hub node,
at least two satellite nodes each comprising a speaker and configured to transmit short-range wireless broadcasts and to establish a short-range wireless communication link with a wearable device, and
at least one wearable device having short-range wireless communication capabilities and comprising a microphone and at least one of at least one sensor and a user input means,
wherein said backend system is configured to transmit downlink data in response to receiving certain uplink data including uplink data indicating at least one of an alert state, a changed physical condition, a changed health state, a changed physical orientation of the person wearing the wearable device and that a call should be established with the wearable device, and
wherein each of the at least one wearable device is having an established short-range wireless communication link with one of the network nodes and is assigned a hub node to have the role of a gatherer node for the wearable device, and wherein the hub node having the gatherer node role for a wearable device is configured to, in response to receiving downlink data from the backend system indicating that a conference call or call should be established with a wearable device the network node is the gatherer node for, initiate the establishment of an internal transmission channel or path within the SON for routing sound recorded by the microphone of the wearable device as audio data to the gatherer node and for routing to the gatherer node incoming audio data of a conference call or call from the gatherer node to another network node so that the audio data can be played out by the speaker of the other network node.

2: The system according to claim 1, wherein the backend system is configured to transmit the downlink data in response to receiving uplink data from the SON indicating to the backend system at least one of an alert state, a changed physical condition, a changed health state, a changed physical orientation of the person wearing the wearable device and that a call should be established with the wearable device.

3: The system according to claim 2, wherein the downlink data received by the SON and the gatherer node is included in at least one of a configuration file which is continuously updated by the backend system and transmitted to the SON, a call initiation request including data for identifying a wearable device which is to participate in a conference call or call, and control data indicating to the hub node having the gatherer node role for a wearable device that the wearable device is to participate in a conference call or call.

4: The system according to claim 1, wherein the backend system is configured to continuously update data associated with the at least one wearable device based on input data and/or sensor data received by the backend system from the SON, and wherein the downlink data which may trigger the initiation of an internal transmission channel or path within the SON is included in a configuration file continuously updated and transmitted by the backend system to the SON.

5: The system according to claim 1, wherein a hub node assigned the gatherer node role for a wearable device is configured to initiate the establishment of an internal transmission channel or path within the SON for the purpose of establishing a conference call or call in which the wearable device is to participate in response to the updated and transmitted configuration file is indicating to the gatherer node at least one of an alert state, a changed physical condition, a changed health state, a changed physical orientation of the person wearing the wearable device and that a call should be established with the wearable device.

6: The system according to claim 1, wherein the other network node is a satellite node and the hub node having the role of a gatherer node for a wearable device is configured to initiate the establishment of an internal transmission channel or path to a satellite node which includes at least one short-range wireless communication link between the satellite node and another network node.

7: The system according to claim 1, wherein the network node having the gatherer node role for a wearable device is configured to use the same internal routing table for initiating the establishment of the internal transmission channel or path for routing audio data obtained by the microphone of the identified wearable device from the other network node to the gatherer node and for routing incoming audio data of a conference call or call to the SON from the gatherer node to the other network node for play out by the speaker of the other network node.

8: The system according to claim 1, wherein the network node having the gatherer node role for a wearable device is configured to determine which one of the network nodes is to receive the audio data incoming to the SON through its speaker based on at least one of signal strength values of wireless short-range signals received from the wearable device which are measured by other network nodes and data shared with the gatherer node role by the hub node having the controller node role for the wearable device.

9: The system according to claim 1, wherein the network node having the gatherer node role for a wearable device is configured to determine that the one network node currently having an established wireless short-range communication link with the wearable device is the network node for receiving incoming audio data over the internal transmission channel or path and be playing out audio data through its speaker.

10: The system according to claim 1, wherein the network node currently having an established wireless short-range communication link with the wearable device is configured to at least one of transmit and forward a request from the gatherer node to the wearable device requesting the wearable device to activate its microphone and then transmit sound recorded by the microphone as audio data to the network node currently having an established wireless short-range communication link with the wearable device.

11: The system according to claim 1, wherein the backend system is configured to initiate the establishment of a conference call or call in which the wearable device is participating and transmit downlink data, e.g. a call initiation request, in response to receiving uplink data in the form of input data and/or sensor data from the SON.

12: The system according to claim 1, wherein the backend system is configured to determine that a conference call or call should be established with the wearable device based on the input data and/or sensor data received from the SON.

13: The system according to claim 1, wherein the uplink data is transmitted to the backend system from the one network node having the controller node role for the wearable device.

14: The system according to claim 1, wherein each of the wearable devices is configured to receiving a request from the network node currently having an established wireless short-range communication link with the wearable device and, in response to receiving the request, activate its microphone to start recording sound data and transmit recorded sound data as audio data to the network node so that the audio data is transmitted over the internal transmission channel or path within the SON to the network node having the gatherer node role for the wearable device.

15: The system according to claim 1, wherein the network node having the gatherer node role for a wearable device is configured to determine which of the network nodes is currently having an established wireless short-range communication link with the wearable device.

16: The system according to claim 1, wherein the network node currently having an established wireless short-range communication link with the wearable device is configured to, in response to receiving a request from the gatherer node, activate its speaker so that incoming audio data from the conference call or call which is transmitted over the internal transmission channel or path is played out by the speaker of the network node currently having an established wireless short-range communication link with the wearable device.

17: The system according to claim 1, wherein the hub node having the gatherer node role for a wearable device is further configured to forward the audio data recorded by the microphone and received over the internal transmission channel or path within the SON to at least one of an external system managing the conference call or call in which an external mobile communication device associated with the wearable device is participating and a mobile communication device associated with the wearable device which is participating in a call with the wearable device.

18: The system according to claim 1, wherein the hub node having the gatherer node role for a wearable device is further configured to, in response to receiving downlink data from the backend system which indicates at least one of an alert state, a changed physical condition, a changed health state, a changed physical orientation of the person wearing the wearable device and that a call should be established with the wearable device, transmit both control data for activating the microphone of the wearable device and control data for activating the speaker of the network node.

19: The system according to claim 1, wherein the other network node is a satellite node the wearable device is currently having a wireless short-range communication link with, and wherein the satellite node is further configured to transmit or forward a request to the wearable device instructing the wearable device to activate its microphone and then start transmitting audio data obtained by the microphone over the wireless short-range communication link.

20: The system according to claim 1, wherein the backend system is configured to transmit the downlink data to the SON at least partly based on at least one of input data obtained by the user input means of the wearable device and sensor data obtained by at least one sensor of the wearable device and which is received by the backend system from the SON.

21: The system according to claim 1, wherein the at least one of input data and sensor data obtained by the wearable device is transmitted to the backend system by the one hub node assigned by the SON to have the controller node role for the wearable device, wherein the controller node is responsible for determining and dynamically changing which of the nodes of the SON is currently having the only established short-range wireless connection between the wearable device and the SON.

22: The system according to claim 1, wherein the downlink data received from the backend system is included in at least one of a configuration file which is continuously updated by the backend system and transmitted from the backend system to the SON, and transmitted as control data transmitted downlink from the backend system to the SON.

23: The system according to claim 1, wherein the downlink data is received by the one hub node assigned by the SON to have the controller node role for the wearable device and responsible for determining and dynamically changing which only one of the nodes of the SON is currently having an established short-range wireless connection with the wearable device, and wherein the downlink data is shared with the hub node having the gatherer node role for the wearable device.

24: The system according to claim 1 wherein the hub node having the gatherer node for a wearable device is further configured to determine which of the nodes is to activate its speaker to play out the incoming audio data from the conference call or call and which is routed over the internal transmission channel or path at least partly based on obtained or received data indicating to the gatherer node which of the nodes is the only node in the SON currently having an established short-range wireless connection with the wearable device.

25: The system according to claim 1, wherein each of the at least one hub node is configured to continuously receive an updated configuration file from the backend system including status data for each of the at least one wearable device, and wherein the hub node having the gatherer node role for a wearable device is configured to initiate the establishment of an internal transmission channel or path within the SON in response to the updated status data for the wearable device is indicating that the wearable device is to participate in a conference call or call.

26: The system according to claim 1, wherein the backend system is configured to, in response to uplink data received from the SON is indicating to the backend system that a conference call or call should be established with one of the at least one wearable device, initiate the establishment of a conference call or call in which the wearable device and a mobile communication device linked to the wearable device in the backend system are to participate.

27: The system according to claim 1, wherein the backend system is configured to initiate the establishment of a conference call or call by transmitting a request to a third-party call facilitator requesting the third-party call facilitator to start a conference call or call in which the wearable device and a mobile communication device linked to the wearable device in the backend system can call into.

28: The system according to claim 1, wherein the backend system is configured to initiate the establishment of a conference call or call by transmitting a request to the hub node having the gatherer node role for a wearable device requesting the gatherer node to activate its two-way audio client to call into the conference call or call.

29: A method for monitoring individuals in a system including a backend system, a self-organizing network (SON) comprising at least three network nodes including at least one hub node, at least two satellite nodes each comprising a speaker and configured to transmit short-range wireless broadcasts and establish a short-range wireless communication link with a wearable device, and at least one wearable device having short-range wireless communication capabilities and comprising a microphone and at least one of at least one sensor and a user input means, the method comprising:
a. assigning one of the at least one hub node to have the role of a gatherer node for a wearable device;
b. transmitting, by the backend system, downlink data to the SON, wherein said downlink data is transmitted in response to receiving, by said backend system transmit, certain uplink data in that said backend system is configured to transmit downlink data in response to receiving certain uplink data including uplink data indicating at least one of an alert state, a changed physical condition, a changed health state, a changed physical orientation of the person wearing the wearable device and that a call should be established with the wearable device, and;
c. receiving, by the hub node having the gatherer node role for a wearable device, the downlink data from the backend system; and
d. initiating, by the hub node having the gatherer node role for the wearable device and in response to the downlink data from the backend system is indicating to the gatherer node that the wearable device is to participate in a conference call or call, the establishment of an internal transmission channel or path within the SON for routing sound recorded by the microphone of the wearable device as audio data to the node having the gatherer node role for the wearable device and for routing to the gatherer node incoming audio data of a conference call or call from the gatherer node to one of the other network nodes so that the audio data can be played out by the speaker of the other network node.

30: The method according to claim 29, further comprising:
forwarding, by the hub node having the gatherer node role for the wearable device, audio data recorded by the microphone of the wearable device and transmitted over the internal transmission channel or path within the SON to at least one of an external system for managing the conference call or call in which the wearable device and a mobile communication device associated with the wearable device is participating and a mobile communication device associated with the wearable device.

31: The method according to claim 29, further comprising:
transmitting, by the hub node currently having the gatherer node role for said wearable device and in response to the downlink data is indicating that a call should be established with the wearable device, control data for activating the microphone of the wearable device.

32: The method according to claim 29, further comprising:
determining, by the hub node having the gatherer node role for the wearable device and based on or in response to the downlink data is indicating that a call should be established with the wearable device, which of the at least three nodes is to activate its speaker to play out audio data received from the conference call or call routed over the internal transmission channel or path.

33: The method according to claim 32, wherein the determining by the gatherer node which of the at least three nodes is to activate its speaker to play out audio data received from the conference call or call is at least partly based on at least one of signal strength values of short-range wireless signals transmitted by the wearable device and which are measured by at least one of the network nodes, and data the wearable device which is collected by at least one other hub node and then shared with the other hub nodes by distribution within the SON.

34: The method according to claim 29, further comprising:
receiving, by the backend system and from the SON, at least one of input data obtained by the user input means of the wearable device and sensor data obtained by at least one sensor of the wearable device; and
transmitting, by the backend system and at least partly based on the received input data and/or sensor data obtained by the user input means and/or at least one sensor of the wearable device, the downlink data to the SON.

35: The method according to claim 34, wherein the at least one of input data and sensor data obtained by the wearable device is transmitted to the backend system by the one hub node assigned by the SON to have the controller node role for the wearable device, wherein said controller node is responsible for determining and dynamically changing which of the nodes of the SON is currently having an established short-range wireless connection with the wearable device.

36: The method according to claim 29, further comprising:
initiating, by the backend system and in response to uplink data received from the SON is indicating to the backend system that a conference call or call should be established with one of the at least one wearable device, a conference call or call in which the wearable device and a mobile communication device linked to the wearable device in the backend system are to participate.

37: The method according to claim 29, further comprising initiating, by the backend system, the establishment of a conference call or call by transmitting a request to a third-party call facilitator requesting the third-party call facilitator to start a conference call or call in which the wearable device and a mobile communication device linked to the wearable device in the backend system can call into.

38: The method according to claim 29, further comprising initiating, by the backend system, the establishment of a conference call or call by transmitting a request to the hub node having the gatherer node role for a wearable device requesting the gatherer node to activate its two-way audio client to call into the conference call or call.
